(19) United States
(12) Patent Application Publication
(10) Pub. No.: US 2024/0358799 A1
(43) Pub. Date: Oct. 31, 2024

(54) METHODS AND COMPOSITIONS FOR REPRODUCTIVE MANAGEMENT OF SHEEP AND GOATS

(71) Applicant: REPROLOGIX, Fort Scott, KS (US)

(72) Inventors: Nathan Wells, Fort Scott, KS (US); Rachel Wells, Fort Scott, KS (US); Shane Morgan, Fort Scott, KS (US); Claire Timlin, Fort Scott, KS (US); Drew Crisler, Fort Scott, KS (US)

(21) Appl. No.: 18/685,172

(22) PCT Filed: Aug. 19, 2022
(86) PCT No.: PCT/US2022/040845

§ 371 Date: Feb. 20, 2024

Related U.S. Application Data
(60) Provisional application No. 63/237,061, filed on Aug. 25, 2021.

(51) Int. Cl. A61K 38/24; A61K 9/00; A61K 31/5575; A61K 31/57; A61K 38/09; A61P 15/08

(52) U.S. Cl. CPC A61K 38/24; A61K 9/0036; A61K 31/5575; A61K 31/57; A61K 38/09; A61P 15/08

(57) ABSTRACT

Compositions and methods for the reproductive management of sheep and goats are provided. More particularly, methods for stimulating follicular maturation and/or synchronizing ovulation in sheep and goats are provided. In some embodiments, the methods and compositions described herein can be used for reproductive management of sheep and goats irrespective of their breeding season, and/or irrespective of estrus detection.

### CROSS-REFERENCE TO RELATED APPLICATION

This application claims priority benefit to U.S. Provisional Patent Application No. 63/237,061, filed on Aug. 25, 2021, the disclosure of which is incorporated by reference herein in its entirety for all purposes.

### FIELD OF THE INVENTION

This disclosure relates to the reproductive management of sheep and goats, and more particularly, to compositions and methods for stimulating follicular maturation and/or synchronizing ovulation therein. In some embodiments, the methods and compositions described herein can be used for reproductive management of sheep and goats irrespective of their breeding season, and/or irrespective of estrus detection.

### BACKGROUND OF THE INVENTION

Controlling the reproductive processes of animals is an important aspect of livestock management. However, in certain animals, aspects of the breeding cycle can be difficult to detect. For example, in some small ruminant animals, such as, e.g., sheep and goats, the onset of estrus can be difficult to determine due to the lack of overt estrous behaviors like those seen in other livestock species, such as cattle. Moreover, some small ruminant animals can experience a short heat cycle (also referred to as a “silent heat”), which is a cycle during which females are not able to become pregnant.

Reproductive management in these animal populations can involve labor-intensive processes, such as the introduction of vasectomized teaser rams into the herd. Teaser rams are typically outfitted with a marking apparatus that marks the female animals who have been mounted, thereby identifying the female animals in whom estrus may have begun. The introduction of teaser rams can also induce the onset of “silent heat” and help the female animals progress into a normal heat cycle, during which a pregnancy can be successfully established.

Some approaches to managing reproductive processes in livestock animals involve the direct administration of hormones and/or hormone-like molecules, such as, e.g., prostaglandins, releasing hormones, gonadotropins, and the like. Such agents can be purified from natural sources, for example, from porcine pituitary glands, or can be chemically synthesized. These agents can be administered to animals via an implant, by intramuscular or subcutaneous injection, by mucosal applications, such as intranasal and intravaginal routes, or per os, where the agent is included in the animals feed and/or water. Some agents can be administered with excipients or delivery systems, which delay or control the release over time to produce more natural or even extended release patterns. See, e.g., U.S. Pat. No. 6,051,558 to Burns, et. al.

Reproductive management of small ruminant animals, such as, e.g., sheep and goats, is further complicated by the seasonal nature of their breeding cycles, with most only engaging in breeding activities at certain times of year. This seasonality condenses breeding activity into a relatively short period of time, during which the animals must be in suitable health for successful breeding. Any complications arising during this time period, for example, poor nutritional status, can mean that the breeding window is missed. Moreover, the difficulty in detecting estrus in sheep and goats further complicates reproductive management, and further increases the chances of a missed breeding window. As such, there is a need for improved methods and compositions that can be used to successfully manage the reproductive processes of sheep and goats, either during or outside of their natural breeding seasons, and irrespective of estrus detection. The present disclosure addresses these and other needs.

### SUMMARY OF THE INVENTION

Aspects of the invention include methods of stimulating follicular maturation in a sheep or goat, the methods comprising: administering a reproductive steroid hormone regimen to the sheep or goat prior to harvesting an ovum from the sheep or goat; and administering a gonadotropin hormone regimen to the sheep or goat prior to harvesting the ovum from the sheep or goat.

In some embodiments, a method further comprises administering a releasing hormone regimen to the sheep or goat prior to harvesting the ovum from the sheep or goat. In some embodiments, the reproductive steroid hormone regimen is administered over a first time period prior to harvesting the ovum from the sheep or goat. In some embodiments, the reproductive steroid hormone regimen comprises a progesterone regimen. In some embodiments, the progesterone regimen comprises a daily dose of progesterone that is administered to the sheep or goat during the first time period. In some embodiments, the daily dose of progesterone ranges from 150 mg to 600 mg. In some embodiments, the daily dose of progesterone ranges from 200 mg to 300 mg. In some embodiments, the daily dose of progesterone is 228 mg.

In some embodiments, the progesterone regimen comprises a continuous dose of progesterone that is administered to the sheep or goat during the first time period. In some embodiments, the continuous dose of progesterone comprises a controlled internal drug release (CIDR) intravaginal device. In some embodiments, the CIDR comprises 150 mg to 450 mg of progesterone. In some embodiments, the CIDR comprises 300 mg of progesterone. In some embodiments, the first time period ranges from 12 to 18 days prior to harvesting the ovum from the sheep or goat. In some embodiments, the first time period ranges from 16 to 18 days prior to harvesting the ovum from the sheep or goat. In some embodiments, the first time period is 17 days prior to harvesting the ovum from the sheep or goat.

In some embodiments, the releasing hormone regimen comprises a single dose of a releasing hormone. In some embodiments, the releasing hormone comprises Gonadotropin Releasing Hormone (GnRH). In some embodiments, the single dose of GnRH ranges from 50 μg to 200 μg. In some embodiments, the single dose of GnRH is 100 μg. In some embodiments, the single dose of GnRH is administered to the sheep or goat 180 hours to 156 hours prior to harvesting the ovum from the sheep or goat. In some embodiments, the single dose of GnRH is administered to the sheep or goat 168 hours prior to harvesting the ovum from the sheep or goat.

In some embodiments, the gonadotropin hormone regimen comprises a gonadotropin hormone selected from the group consisting of: follicle stimulating hormone (FSH), luteinizing hormone (LH), human chorionic gonadotropin (hCG), and any combination thereof. In some embodiments, the gonadotropin hormone regimen comprises FSH. In some embodiments, the FSH is administered over a second time period prior to harvesting the ovum from the sheep or goat. In some embodiments, the second time period ranges from 120 to 24 hours prior to harvesting the ovum from the sheep or goat. In some embodiments, the second time period ranges from 120 to 36 hours prior to harvesting the ovum from the sheep or goat.

In some embodiments, the FSH comprises a dose that ranges from 160 μg to 240 μg. In some embodiments, the FSH dose is 200 μg. In some embodiments, the FSH is administered continuously over the second time period. In some embodiments, the FSH is administered via twice daily doses that are administered 10-14 hours apart over the second time period. In some embodiments, the twice daily doses are administered in a decreasing manner over the second time period.

In some embodiments, the gonadotropin hormone regimen comprises a single dose of LH. In some embodiments, the single dose of LH ranges from 12 mg to 50 mg. In some embodiments, the single dose of LH is 25 mg. In some embodiments, the single dose of LH is administered to the sheep or goat 180 hours to 156 hours prior to harvesting the ovum from the sheep or goat. In some embodiments, the single dose of LH is administered to the sheep or goat 168 hours prior to harvesting the ovum from the sheep or goat. In some embodiments, the gonadotropin hormone regimen comprises a single dose of hCG. In some embodiments, the single dose of hCG ranges from 50 IU to 200 IU. In some embodiments, the single dose of hCG is 100 IU. In some embodiments, the single dose of hCG ranges from 1,750 IU to 4,000 IU. In some embodiments, the single dose of hCG is 2,500 IU.

In some embodiments, the single dose of hCG is administered to the sheep or goat 180 hours to 156 hours prior to harvesting the ovum from the sheep or goat. In some embodiments, the single dose of hCG is administered to the sheep or goat 168 hours prior to harvesting the ovum from the sheep or goat.

In some embodiments, the methods further comprise administering a prostaglandin regimen to the sheep or goat prior to harvesting the ovum from the sheep or goat. In some embodiments, the prostaglandin regimen comprises a single dose of prostaglandin that is administered 4 to 6 days prior to harvesting the ovum from the sheep or goat. In some embodiments, the single dose of prostaglandin is administered 5 days prior to harvesting the ovum from the sheep or goat. In some embodiments, the prostaglandin is Cloprostenol or Dinoprost. In some embodiments, the prostaglandin is Cloprostenol, and the single dose of Cloprostenol ranges from 125 mg to 625 mg. In some embodiments, the single dose of Cloprostenol is 375 mg. In some embodiments, the prostaglandin is Dinoprost, and the single dose of Dinoprost ranges from 7.5 mg to 20 mg. In some embodiments, the single dose of Dinoprost is 15 mg.

In some embodiments, the methods further comprise harvesting the ovum from the sheep or goat. In some embodiments, the methods further comprise terminating the progesterone regimen. In some embodiments, the methods further comprise fertilizing the ovum to generate an embryo.

In some embodiments, the methods are conducted during a breeding season of the sheep or goat. In some embodiments, the methods are conducted outside of a breeding season of the sheep or goat.

Aspects of the invention include methods of stimulating follicular maturation in a sheep or goat, the methods comprising: administering a continuous dose of progesterone to the sheep or goat for 17 days prior to harvesting an ovum from the sheep or goat, wherein the continuous dose of progesterone is administered via a controlled internal drug release (CIDR) intravaginal device comprising 300 mg of progesterone: administering a releasing hormone regimen to the sheep or goat, comprising a single dose of 100 μg of gonadotropin releasing hormone (GnRH) administered 168 hours prior to harvesting the ovum from the sheep or goat: and administering a gonadotropin hormone regimen to the sheep or goat, comprising a continuous dose of 160 to 240 μg of follicle stimulating hormone (FSH) administered over a time period ranging from 120-24 hours prior to harvesting the ovum from the sheep or goat; and administering a single dose of prostaglandin to the sheep or goat 5 days prior to harvesting the ovum from the sheep or goat, wherein the single dose of prostaglandin is selected from the group consisting of: 375 g of Cloprostenol and 15 mg of Dinoprost.

Aspects of the invention include methods of generating a fertilized embryo from a sheep or goat, the methods comprising: administering a continuous dose of progesterone to the sheep or goat for 17 days prior to harvesting an ovum from the sheep or goat,wherein the continuous dose of progesterone is administered via a controlled internal drug release (CIDR) intravaginal device comprising 300 mg of progesterone; administering a releasing hormone regimen to the sheep or goat, comprising a single dose of 100 μg of gonadotropin releasing hormone (GnRH) administered 168 hours prior to harvesting the ovum from the sheep or goat; and administering a gonadotropin hormone regimen to the sheep or goat,comprising a continuous dose of 160 to 240 μg of follicle stimulating hormone (FSH) administered over a time period ranging from 120-24 hours prior to harvesting the ovum from the sheep or goat: administering a single dose of prostaglandin to the sheep or goat 5 days prior to harvesting the ovum from the sheep or goat, wherein the single dose of prostaglandin is selected from the group consisting of: 375 μg of Cloprostenol and 15 mg of Dinoprost; harvesting the ovum from the sheep or goat: and fertilizing the ovum to generate an embryo.

Aspects of the invention include methods of synchronizing ovulation in a plurality of sheep or goats, the methods comprising: administering a reproductive steroid regimen to the sheep or goats; and administering a gonadotropin hormone regimen to the sheep or goats.

In some embodiments, the reproductive steroid hormone regimen is administered over a first time period prior to ovulation synchronization. In some embodiments, the reproductive steroid hormone regimen comprises a progesterone regimen. In some embodiments, the progesterone regimen comprises a daily dose of progesterone that is administered to the sheep or goats during the first time period. In some embodiments, the daily dose of progesterone ranges from 150 mg to 600 mg. In some embodiments, the daily dose of progesterone ranges from 200 mg to 300 mg. In some embodiments, the daily dose of progesterone is 228 mg.

In some embodiments, the progesterone regimen comprises a continuous dose of progesterone that is administered to the sheep or goats during the first time period. In some embodiments, the continuous dose of progesterone comprises a controlled internal drug release (CIDR) intravaginal device. In some embodiments, the CIDR comprises 150 mg to 450 mg of progesterone. In some embodiments, the CIDR comprises 300 mg of progesterone. In some embodiments, the first time period ranges from 12 to 18 days prior to ovulation synchronization. In some embodiments, the first time period ranges from 16 to 18 days prior to ovulation synchronization. In some embodiments, the first time period is 17 days prior to ovulation synchronization.

In some embodiments, the gonadotropin hormone regimen comprises a gonadotropin hormone selected from the group consisting of: equine chorionic gonadotropin (eCG), human chorionic gonadotropin (hCG), and any combination thereof. In some embodiments, the gonadotropin hormone regimen comprises administering a single dose of eCG to the sheep or goats. In some embodiments, the single dose of eCG ranges from 100 to 400 IU. In some embodiments, the single dose of eCG is 200 IU. In some embodiments, the gonadotropin hormone regimen comprises administering a single dose of hCG to the sheep or goats. In some embodiments, the single dose of hCG ranges from 50 to 200 IU. In some embodiments, the single dose of hCG is 100 IU. In some embodiments, the single dose of hCG ranges from 1,750 IU to 4,000 IU. In some embodiments, the single dose of hCG is 2,500 IU. In some embodiments, the gonadotropin hormone regimen comprises a single dose of eCG ranging from 100 to 400 IU and a single dose of hCG ranging from 50 to 200 IU. In some embodiments, the gonadotropin hormone regimen comprises a single dose of 200 IU of eCG and a single dose of 100 IU of hCG. In some embodiments, the gonadotropin hormone regimen is administered to the sheep or goats 12 to 36 hours prior to ovulation synchronization. In some embodiments, the gonadotropin hormone regimen is administered to the sheep or goats 24 hours prior to ovulation synchronization.

In some embodiments, the methods further comprise administering a prostaglandin regimen to the sheep or goats prior to ovulation synchronization. In some embodiments, the prostaglandin regimen comprises a single dose of prostaglandin that is administered 12 to 36 hours prior to ovulation synchronization. In some embodiments, the single dose of prostaglandin is administered 24 hours prior to ovulation synchronization. In some embodiments, the prostaglandin is Cloprostenol or Dinoprost. In some embodiments, the prostaglandin is Cloprostenol, and the single dose of Cloprostenol ranges from 125 μg to 625 μg. In some embodiments, the single dose of Cloprostenol is 375 μg. In some embodiments, the prostaglandin is Dinoprost, and the single dose of Dinoprost ranges from 7.5 mg to 20 mg. In some embodiments, the single dose of Dinoprost is 15 mg.

In some embodiments, the methods result in a 90% or greater rate of synchronization of ovulation between the sheep or goats.

In some embodiments, the methods further comprise verifying ovulation in one or more of the sheep or goats. In some embodiments, verifying ovulation comprises detecting a corpus luteum in an ovary of one or more of the sheep or goats. In some embodiments, the methods further comprise detecting estrus in one or more of the sheep or goats.

In some embodiments, the methods further comprise transferring an in vitro fertilized embryo into one or more of the sheep or goats. In some embodiments, the in vitro fertilized embryo is transferred into the sheep or goats 7 to 8 days after ovulation synchronization.

In some embodiments, the methods further comprise terminating the progesterone regimen upon ovulation synchronization.

Aspects of the invention include methods of synchronizing ovulation in a plurality of sheep or goats, the methods comprising: administering a continuous dose of progesterone to the sheep or goats for 17 days prior to synchronization of ovulation, wherein the continuous dose of progesterone is administered via a controlled internal drug release (CIDR) intravaginal device comprising 300 mg of progesterone; administering a gonadotropin regimen to the sheep or goats 24 hours prior to synchronization of ovulation, comprising a single dose of 200 IU of eCG and a single dose of 100 IU of hCG; and administering a single dose of prostaglandin to the sheep or goats 24 hours prior to synchronization of ovulation, wherein the single dose of prostaglandin is selected from the group consisting of: 375 μg of Cloprostenol and 15 mg of Dinoprost.

Aspects of the invention include methods of establishing a pregnancy in a sheep or goat, the methods comprising: administering a continuous dose of progesterone to the sheep or goat for 17 days prior to an embryo transfer procedure, wherein the continuous dose of progesterone is administered via a controlled internal drug release (CIDR) intravaginal device comprising 300 mg of progesterone; administering a gonadotropin regimen to the sheep or goat 24 hours prior to the embryo transfer procedure, comprising a single dose of 200 IU of eCG and a single dose of 100 IU of hCG; administering a single dose of prostaglandin to the sheep or goat 24 hours prior to the embryo transfer procedure, wherein the single dose of prostaglandin is selected from the group consisting of: 375 μg of Cloprostenol and 15 mg of Dinoprost; and conducting the embryo transfer procedure, wherein a fertilized embryo is transferred into the sheep or goat to establish the pregnancy.

Aspects of the invention include methods of establishing a pregnancy in a sheep or goat, the methods comprising: (i) stimulating follicular maturation in a donor sheep or goat by: administering a continuous dose of progesterone to the donor sheep or goat for 17 days prior to harvesting an ovum from the donor sheep or goat, wherein the continuous dose of progesterone is administered via a controlled internal drug release (CIDR) intravaginal device comprising 300 mg of progesterone; administering a releasing hormone regimen to the donor sheep or goat, comprising a single dose of 100 μg of gonadotropin releasing hormone (GnRH) administered 168 hours prior to harvesting the ovum from the donor sheep or goat; administering a gonadotropin hormone regimen to the donor sheep or goat, comprising a continuous dose of 160 to 240 μg of follicle stimulating hormone (FSH) administered over a time period ranging from 120 to 24 hours prior to harvesting the ovum from the donor sheep or goat; and administering a single dose of prostaglandin to the donor sheep or goat 5 days prior to harvesting the ovum from the donor sheep or goat, wherein the single dose of prostaglandin is selected from the group consisting of: 375 μg of Cloprostenol and 15 mg of Dinoprost; (ii) synchronizing ovulation in the donor sheep or goat and a recipient sheep or goat by: administering a continuous dose of progesterone to the recipient sheep or goat for 17 days prior to synchronization of ovulation, wherein the continuous dose of progesterone is administered via a controlled internal drug release (CIDR) intravaginal device comprising 300 mg of progesterone; administering a gonadotropin regimen to the recipient sheep or goat 24 hours prior to synchronization of ovulation, comprising a single dose of 200 IU of eCG; and administering a single dose of prostaglandin to the recipient sheep or goat 24 hours prior to synchronization of ovulation, wherein the single dose of prostaglandin is selected from the group consisting of: 375 μg of Cloprostenol and 15 mg of Dinoprost; (iii) harvesting an ovum from the donor sheep or goat; (iv) fertilizing the ovum to generate a fertilized embryo; and (v) transferring the fertilized embryo into the recipient sheep or goat to establish a pregnancy.

Aspects of the invention include methods of establishing a pregnancy in a sheep or goat, the methods comprising: (i) stimulating follicular maturation in a donor sheep or goat by: administering a continuous dose of progesterone to the donor sheep or goat for 17 days prior to harvesting an ovum from the donor sheep or goat, wherein the continuous dose of progesterone is administered via a controlled internal drug release (CIDR) intravaginal device comprising 300 mg of progesterone; administering a releasing hormone regimen to the donor sheep or goat, comprising a single dose of 100 μg of gonadotropin releasing hormone (GnRH) administered 168 hours prior to harvesting the ovum from the donor sheep or goat; administering a gonadotropin hormone regimen to the donor sheep or goat, comprising a continuous dose of 160 to 240 μg of follicle stimulating hormone (FSH) administered over a time period ranging from 120 to 24 hours prior to harvesting the ovum from the donor sheep or goat; and administering a single dose of prostaglandin to the donor sheep or goat 5 days prior to harvesting the ovum from the donor sheep or goat, wherein the single dose of prostaglandin is selected from the group consisting of: 375 μg of Cloprostenol and 15 mg of Dinoprost; (ii) synchronizing ovulation in the donor sheep or goat and a recipient sheep or goat by: administering a continuous dose of progesterone to the recipient sheep or goat for 17 days prior to synchronization of ovulation, wherein the continuous dose of progesterone is administered via a controlled internal drug release (CIDR) intravaginal device comprising 300 mg of progesterone: administering a gonadotropin regimen to the recipient sheep or goat 24 hours prior to synchronization of ovulation, comprising a single dose of 100 IU of hCG; and administering a single dose of prostaglandin to the recipient sheep or goat 24 hours prior to synchronization of ovulation, wherein the single dose of prostaglandin is selected from the group consisting of: 375 μg of Cloprostenol and 15 mg of Dinoprost; (iii) harvesting an ovum from the donor sheep or goat; (iv) fertilizing the ovum to generate a fertilized embryo; and (v) transferring the fertilized embryo into the recipient sheep or goat to establish a pregnancy.

Aspects of the invention include methods of establishing a pregnancy in a sheep or goat, the methods comprising: (i) stimulating follicular maturation in a donor sheep or goat by: administering a continuous dose of progesterone to the donor sheep or goat for 17 days prior to harvesting an ovum from the donor sheep or goat, wherein the continuous dose of progesterone is administered via a controlled internal drug release (CIDR) intravaginal device comprising 300 mg of progesterone; administering a releasing hormone regimen to the donor sheep or goat, comprising a single dose of 100 μg of gonadotropin releasing hormone (GnRH) administered 168 hours prior to harvesting the ovum from the donor sheep or goat; administering a gonadotropin hormone regimen to the donor sheep or goat, comprising a continuous dose of 160 to 240 μg of follicle stimulating hormone (FSH) administered over a time period ranging from 120 to 24 hours prior to harvesting the ovum from the donor sheep or goat; and administering a single dose of prostaglandin to the donor sheep or goat 5 days prior to harvesting the ovum from the donor sheep or goat, wherein the single dose of prostaglandin is selected from the group consisting of: 375 μg of Cloprostenol and 15 mg of Dinoprost; (ii) synchronizing ovulation in the donor sheep or goat and a recipient sheep or goat by: administering a continuous dose of progesterone to the recipient sheep or goat for 17 days prior to synchronization of ovulation, wherein the continuous dose of progesterone is administered via a controlled internal drug release (CIDR) intravaginal device comprising 300 mg of progesterone; administering a gonadotropin regimen to the recipient sheep or goat 24 hours prior to synchronization of ovulation, comprising a single dose of 200 IU of eCG and a single dose of 100 IU or hCG; and administering a single dose of prostaglandin to the recipient sheep or goat 24 hours prior to synchronization of ovulation, wherein the single dose of prostaglandin is selected from the group consisting of: 375 μg of Cloprostenol and 15 mg of Dinoprost; (iii) harvesting an ovum from the donor sheep or goat; (iv) fertilizing the ovum to generate a fertilized embryo; and (v) transferring the fertilized embryo into the recipient sheep or goat to establish a pregnancy.

In some embodiments, administering the gonadotropin hormone regimen to the donor sheep or goat comprises administering a continuous dose of 200 μg of FSH to the donor sheep or goat. In some embodiments, the methods result in a pregnancy rate of 50% or greater in a plurality of recipient animals. In some embodiments, the methods result in a pregnancy rate in the plurality of recipient animals that ranges from 55% to 80%. In some embodiments, the methods result in a pregnancy rate in the plurality of recipient animals of 63.5%.

These and further aspects will be further explained in the rest of the disclosure, including the Examples.

### BRIEF DESCRIPTION OF THE DRAWINGS

FIG. 1 is a graph showing known international average pregnancy rate for small ruminant IVP embryos, in comparison to average pregnancy rate achieved using the methods described herein.

### DETAILED DESCRIPTION OF THE PREFERRED EMBODIMENTS

The practice of the present invention will employ, unless otherwise indicated, conventional techniques of animal husbandry, which are within the skill of the art. Such techniques are explained fully in the literature, such as. Banerjee. G.C., 2018. A textbook of animal husbandry. Oxford and IBH publishing: and Blakely. J, and Bade, D.H., 1990. The science of animal husbandry. Prentice-Hall Inc., the disclosures of which are incorporated by reference herein in their entireties.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

All references cited throughout the disclosure, including patent applications and publications, are incorporated by reference herein in their entirety.

### I. Definitions

By “comprising” it is meant that the recited elements are required in the composition/method/kit, but other elements may be included to form the composition/method/kit etc. within the scope of the claim.

By “consisting essentially of”, it is meant a limitation of the scope of composition or method described to the specified materials or steps that do not materially affect the basic and novel characteristic(s) of the subject invention.

By “consisting of”, it is meant the exclusion from the composition, method, or kit of any element, step, or ingredient not specified in the claim.

The term “regimen” as used herein refers to any combination of an amount and a frequency of administration of a drug or agent, including, without limitation, a single dose, a daily dose, a continuous dose, or the like, using a fixed or variable amount of the drug or agent. As such, a “drug A regimen” could comprise any amount of drug A (e.g., 10 mg, 100 mg, etc.), administered at any dosing frequency (e.g., once daily, twice daily, continuously over a designated time period (e.g., 5 days, 10 days), etc.).

The terms “compound”, “agent”, “active agent”, “hormone” and “drug” can be used interchangeably herein to refer to a therapeutic composition as described herein. In some cases, the terms “additional compound”, “additional agent”, or “additional therapeutic agent” can be used interchangeably to refer to other active compounds, agents, or therapeutic compositions that may be used in a composition described herein.

The terms “administer,” “administering”, “administration,” and the like, as used herein, refer to methods that can be used to enable delivery of compounds or compositions to a desired site of biological action. These methods can include oral administration (administration per os), intraduodenal administration, parenteral administration (including intravenous, subcutaneous, intrathecal, intraperitoneal, intramuscular, or intravascular infusion), topical, vaginal and rectal administration.

In some embodiments, administration can be vaginal administration. Vaginal administration can include administering to any surface of a vagina. In some cases, vaginal administration can be intravaginal administration. Vaginal administration can include applying a composition described herein to a vagina using an applicator. Vaginal administration can include administering intravaginally, administering topically to a vagina, or a combination of such administrations. Administering vaginally can also include administering via a carrier, such as a patch, a suppository, an implantable drug reservoir (e.g., a CIDR intravaginal device), a tablet and the like.

The terms “pharmaceutically acceptable salt” or simply “salt” as used herein, can refer to a salt that retains at least some of the biological effectiveness of the free acids and bases of the specified compound. In some instances, a salt is not biologically or otherwise undesirable. In some embodiments, a compound disclosed herein can comprise acidic or basic groups and therefore can react with any of a number of inorganic and/or organic bases, and inorganic and/or organic acids, to form a pharmaceutically acceptable salt. In some embodiments, a salt can be prepared in situ during the final isolation and purification of a compound, or by separately reacting a purified compound in its free base form with a suitable organic or inorganic acid, and isolating the salt thus formed.

The term “pharmaceutical composition.” or simply “composition” as used herein, can refer to an active agent, optionally mixed with at least one pharmaceutically acceptable chemical component, such as a carrier, a stabilizer, a diluent, a dispersing agent, a suspending agent, a thickening agent, an excipient, a bioadhesive, and the like. A dispensing agent can be an emulsion that can comprise a substantially uniform mixture of an organic phase and an aqueous phase. An organic phase and an aqueous phase can comprise components dissolved or suspended therein. While exemplary embodiments may describe a localization of a component with a single phase, it is understood that any component can be present in either phase.

The terms “co-administration”, “administered in combination with” and their grammatical equivalents, as used herein, encompass administration of selected compositions to a single subject, and can include dosing regimens in which the compositions can be administered by the same or different routes of administration, or at the same or different times. In some embodiments, a composition disclosed herein can be co-administered with other agents. These terms can encompass administration of two or more agents to an animal so that both agents and/or their metabolites can be present in the animal at the same time. They can include simultaneous administration in separate compositions, administration at different times in separate compositions, and/or administration in a composition in which both agents can be present. Thus, in some embodiments, a compound and another agent(s) can be administered in a single composition. In some embodiments, a compound and another agent(s) can be admixed in the composition.

The term “single dose” as used herein refers to a dose of a compound that is administered in full at a single point in time.

The term “daily dose” as used herein refers to a dose of a compound that is administered in full on a recurring basis, approximately once every 24 hours, +/−4 hours.

The term “twice daily dose” as used herein refers to a dose of a compound that is administered in full on a recurring basis, approximately once every 12 hours, +/−4 hours.

The term “continuous dose” as used herein refers to a dose of a compound that is administered over a time period, such that administration of the compound is actively taking place over the time period. Non-limiting examples of a continuous dose include: administration of a liquid solution comprising a specified concentration of a drug or agent via intravenous (iv) infusion over a designated time period, and implantation or placement of a controlled internal release drug device (e.g., a CIDR intravaginal device comprising a reservoir of the compound) for a designated time period.

The terms “controlled internal drug release intravaginal device” and “CIDR intravaginal device” as used interchangeably herein refer to device that comprises a reservoir comprising a fixed amount of a compound (e.g., a liquid, solid or semisolid reservoir) and is configured to be placed vaginally in a subject (e.g., a sheep or a goat) to release the compound, which is absorbed by the subject across a mucosal surface of the vagina over a period of time. In some embodiments, a CIDR intravaginal device comprises a designated amount of a compound in the reservoir (e.g., 300 mg of progesterone), which is released over a period of hours or days, and is absorbed by the subject. In some embodiments, a CIDR intravaginal device can be recharged by refilling or replacing the reservoir.

The term “releasing hormone” as used herein refers to a hormone whose primary function is to control or modulate the release of other hormones. One non-limiting example of a releasing hormone is Gonadotropin Releasing Hormone (GnRH).

The terms “gonadotropin hormone” and “gonadotropin” as used interchangeably herein refer to hormones secreted by gonadotropic cells of the anterior pituitary gland, which generally regulate growth, sexual development, and reproductive functions. Non-limiting examples of gonadotropins include follicle stimulating hormone (FSH), luteinizing hormone (LH), equine chorionic gonadotropin (cCG), and human chorionic gonadotropin (hCG).

The terms “ovulation synchronization” and “synchronization of ovulation” as used interchangeably herein refer to synchronizing the timing of release of an egg from an ovary in two or more different subjects (e.g., two or more different small ruminant animals) to occur within the same 24-hour period.

The term “follicular maturation” as used herein refers to the development and maturation of an ovarian follicle comprising an immature oocyte in an ovary of a subject (e.g., a small ruminant animal).

The terms “International Units” and “IU” as used interchangeably herein refer to a unit that measures a biological effect of a compound (e.g., a hormone) rather than the mass of the compound. IUs are generally used to measure biologically active substances that can have different activity levels from lot to lot. The precise amount of a compound that constitutes one IU differs from substance to substance, and is typically established by international agreement for each substance. One of ordinary skill in the art will readily appreciate that for a given substance, IUs can be converted to milligrams, and vice versa.

### II. Detailed Description

Aspects of the invention relate to the reproductive management of sheep and goats, and more particularly, to compositions and methods for stimulating follicular maturation and/or synchronizing ovulation therein. In some embodiments, the methods and compositions described herein can be used for reproductive management of sheep and goats irrespective of their breeding season, and/or irrespective of estrus detection.

Attempted hormonal control of the estrus period and ovulation in livestock animals is well described in the literature. Such techniques have been described using more than one steroid, gonadotropin, prostaglandin, or their analogs in series or in combination under various timing conditions, depending on the livestock animal under study. For example, injectable and oral progesterone and progestogens are described in Ulberg et al (1951) J. Animal Sci. 10, 665-671) and Gerrits et al., (1963) J. Animal Sci. 21, 1022-1025). Altrenogest is described in Martinat-Botte et al., 1985 Martinatt-Botte, F., Bariteau, F., Badouard, B. and Terqui, M. (1985) J. Reprod. Fert. Suppl. 33, 211-228); altrenogest with PMSG and GnRH/hCG is described in Busch et al., (1992) Monatshefte fur Veteriarmedizin 47, 307-316); prostaglandins are described in Jackson and Hutchinson, Veterinary Record 106 33-34; and methallibure, PMSG and hCG are described in Paige et al., (1968) Veterinary Record 83, 136-142 and F. De Rensis et al., (2003) Animal Reproduction Science 76:245-250. These techniques have either met with limited success (progestogens), failed (prostaglandins), been banned from the market (methallibure) or require daily oral dosing (altrenogest), multiple injections (estradiol, progesterone) or combinations of drugs (PMSG, hCG, GnRH) coupled with continued estrus detection in order to create detectable breeding efficiencies.

The present disclosure provides various protocols and dosing regimens that can be used to stimulate follicular maturation in a donor animal, as well as protocols and dosing regimens that can be used to synchronize ovulation in a plurality of animals (e.g., a donor and a recipient animal; a plurality of recipient animals, etc). Aspects of the disclosure also relate to combinations of these donor and recipient protocols, which can be used to establish a pregnancy in an animal. Aspects of the disclosure include methods and compositions that can be used to successfully achieve stimulation of follicular maturation, synchronization of ovulation, and pregnancy in a sheep or goat irrespective of the breeding season of the animal, and/or irrespective of estrus detection.

### Compositions:

Aspects of the invention include compositions that can be administered to a subject (e.g., a sheep or a goat) for reproductive management, which are described further below.

### Reproductive Steroid Hormones:

Aspects of the invention include methods that involve administration of a reproductive steroid hormone to an animal (e.g., a sheep or a goat). Non-limiting examples of reproductive steroid hormones include progesterone and estradiol.

In some embodiments, a composition comprises progesterone. Progesterone is commercially available in various formats, including, without limitation, progesterone isolated from natural sources, synthetically produced progesterone, or any pharmaceutically acceptable salt thereof, or any analogue thereof. Non-limiting examples of progesterone include Progesterone USP 36, produced by AX Pharmaceutical Corp; progesterone implants, such as SYNOVEX S® and EAZI-BREED CIDR®; and synthetic progestin feed additives (melengestrol acetate (MGAR), HEIFERMAX®), produced by Pfizer, Inc.). In some embodiments, progesterone can be administered via a progesterone-releasing intravaginal device (PRID).

In some embodiments, a composition comprises a progesterone dose that ranges from about 50 mg up to about 750 mg, such as 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 216, 217, 218, 219, 220, 221, 222, 223, 224, 225, 226, 227, 228, 229, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740 or 745 mg.

In some embodiments, a composition comprises estradiol. Estradiol is commercially available in various formats, including, without limitation, estradiol isolated from natural sources, synthetically produced estradiol, or any pharmaceutically acceptable salt thereof, or any analogue thereof. Non-limiting examples of estradiol include YUVAFEM® (Amneal Pharmaceuticals, Inc.) and ESTROGEL® (Ascend Therapeutics, Inc.).

In some embodiments, a composition comprises an estradiol dose that ranges from about 100 mg up to about 400 micrograms (μg), such as 105, 110, 115, 120, 125, 130, 135, 140, 145, 150, 155, 160, 165, 170, 175, 180, 185, 190, 195, 200, 205, 210, 215, 216, 217, 218, 219, 220, 221, 222, 223, 224, 225, 226, 227, 228, 229, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, or 395 μg.

### Gonadotropins:

In some embodiments, a composition comprises a gonadotropin. Gonadotropins are hormones produced by the anterior lobe of the pituitary gland and include, for example, follicle stimulating hormone (FSH), luteinizing hormone (LH) and chorionic gonadotropins (e.g., equine chorionic gonadotropin (eCG) and human chorionic gonadotropin (hCG)). A number of different preparations of gonadotropins are commercially available, including, e.g., Chorulon, Folltropin-V, P.G. 600 (mixture of eCG and hCG), Lutropin-V and others.

In some embodiments, a composition comprises an FSH dose that ranges from about 5 μg up to about 750 μg, such as 5.5, 6, 6.5, 7, 7.5, 8, 8.5, 9, 9.5, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 216, 217, 218, 219, 220, 221, 222, 223, 224, 225, 226, 227, 228, 229, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740 or 745 μg.

In some embodiments, a composition comprises an FSH dose that ranges from about 1 mg up to about 50 mg, such as 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 mg.

In some embodiments, a composition comprises an FSH dose that ranges from about 10 IU up to about 500 IU, such as 11, 12, 13, 14, 15, 16, 17, 18, 19, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, or 495 IU.

In some embodiments, a composition comprises an LH dose that ranges from about 5 μg up to about 750 μg, such as 5.5, 6, 6.5, 7, 7.5, 8, 8.5, 9, 9.5, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 216, 217, 218, 219, 220, 221, 222, 223, 224, 225, 226, 227, 228, 229, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740 or 745 μg.

In some embodiments, a composition comprises an LH dose that ranges from about 1 mg up to about 50 mg, such as 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 mg.

In some embodiments, a composition comprises an LH dose that ranges from about 10 IU up to about 500 IU, such as 11, 12, 13, 14, 15, 16, 17, 18, 19, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, or 495 IU.

In some embodiments, a composition comprises an eCG dose that ranges from about 1 mg up to about 750 mg, such as 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 216, 217, 218, 219, 220, 221, 222, 223, 224, 225, 226, 227, 228, 229, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740 or 745 mg.

In some embodiments, a composition comprises an eCG dose that ranges from about 10 IU up to about 500 IU, such as 11, 12, 13, 14, 15, 16, 17, 18, 19, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, or 495 IU.

In some embodiments, a composition comprises an hCG dose that ranges from about 1 mg up to about 750 mg, such as 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 216, 217, 218, 219, 220, 221, 222, 223, 224, 225, 226, 227, 228, 229, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740 or 745 mg.

In some embodiments, a composition comprises an hCG dose that ranges from about 10 IU up to about 500 IU, such as 11, 12, 13, 14, 15, 16, 17, 18, 19, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, or 495 IU.

In some embodiments, a composition comprises an hCG dose that ranges from about 1,500 IU up to about 4,000 IU, such as 1,550, 1,600, 1,650, 1,700, 1,750, 1,800, 1,850, 1,900, 1,950, 2,000, 2,050, 2,100, 2,150, 2,200, 2,250, 2,300, 2,350, 2,400, 2,450, 2,500, 2,550, 2,600, 2,650, 2,700, 2,750, 2,800, 2,850, 2,900, 2,950, 3,000, 3,050, 3,100, 3,150, 3,200, 3,250, 3,300, 3,350, 3,400, 3,450, 3,500, 3,550, 3,600, 3,650, 3,700, 3,750, 3,800, 3,850, 3,900 or 3,950 IU.

### Releasing Hormones:

In some embodiments, a composition comprises one or more releasing hormones. Releasing hormones are hormones whose primary function is to modulate the release of other hormones. One non-limiting example of a releasing hormone is gonadotropin-releasing hormone (GnRH).

GnRH is commercially available in various formats, including, without limitation, GnRH isolated from natural sources, synthetically produced GnRH, or any pharmaceutically acceptable salt thereof, or any analogue thereof. Non-limiting examples of GnRH that can be used in aspects of the disclosure include Gonadorelin (a synthetic analogue of GnRH, tradenames CYSTORELIN®); FERTAGYL®; GONABREED®; FACTREL®) and Buserelin (a synthetic analogue of GnRH, tradename RECEPTAL®).

In some embodiments, a composition comprises a GnRH dose that ranges from about 10 μg up to about 950 μg, such as 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940 or 945 μg.

In some embodiments, a composition comprises an GnRH dose that ranges from about 1 mg up to about 750 mg, such as 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 216, 217, 218, 219, 220, 221, 222, 223, 224, 225, 226, 227, 228, 229, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740 or 745 mg.

### Prostaglandins:

In some embodiments, a composition comprises a prostaglandin. Prostaglandins are commercially available in various formats, including, without limitation, prostaglandin isolated from natural sources, synthetically produced prostaglandin, or any pharmaceutically acceptable salt thereof, or any analogue thereof. Non-limiting examples of prostaglandin that can be used in aspects of the disclosure include Cloprostenol (a synthetic analogue of prostaglandin F2α, tradenames CYCLOMATE®; ESTRUMATE®; SYNCHSURE®; ESTROPLAN®; and Dinoprost (a synthetic analogue of prostaglandin F2α, tradename LUTALYSE®).

In some embodiments, a composition comprises a prostaglandin dose that ranges from about 5 μg up to about 995 μg, such as 10, 20, 30, 40, 50, 60, 70, 80, 90, 100, 110, 120, 125, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 625, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980 or 990 μg.

In some embodiments, a composition comprises a prostaglandin dose that ranges from about 5 mg up to about 750 mg, such as 6, 6.5, 7, 7.5, 8, 8.5, 9, 9.5, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 216, 217, 218, 219, 220, 221, 222, 223, 224, 225, 226, 227, 228, 229, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740 or 745 mg.

### Methods:

Aspects of the invention include methods of administering one or more compositions as described herein, or combinations thereof, to a subject (e.g., a sheep or a goat) for reproductive management. Methods in accordance with embodiments of the invention include donor protocols that can be used to stimulate follicular maturation in a subject, recipient protocols that can be used to synchronize ovulation in a plurality of subjects, and pregnancy protocols that can be used to establish a pregnancy in a subject.

In some embodiments, the methods can be performed on a small ruminant animal (e.g., a sheep or a goat), and can be used to achieve reproductive management of the animal irrespective of its breeding season, and irrespective of estrus detection. Some smaller ruminant animals (e.g., sheep and goats) exhibit seasonal breeding patterns, which can limit reproductive management options to certain times of year. Additionally, estrus can be difficult to detect in these animals, which further complicates reproductive management techniques. In some embodiments, the methods provided herein can be used irrespective of breeding season, i.e., can be used during or outside of a natural breeding season of the animal, to successfully manage its reproductive activity. Furthermore, in some embodiments, the methods provided herein can be used without having to determine or confirm estrus in the animal.

### Donor Protocol

Aspects of the invention include donor protocols (methods) that can be used to stimulate follicular maturation in a subject (e.g., a sheep or a goat). In some embodiments, a donor protocol involves administering a reproductive steroid hormone regimen (e.g., a progesterone regimen or an estradiol regimen) and a gonadotropin regimen to the subject prior to harvesting an ovum from the subject. In certain embodiments, a donor protocol further comprises administering a releasing hormone regimen to the subject prior to harvesting the ovum from the subject.

Aspects of the invention include methods that involve administering one or more dosing regimens to a subject over a specified time period prior to harvesting an ovum from the subject. For example, in one embodiment, a donor protocol involves administering a reproductive steroid hormone regimen (e.g., a progesterone regimen) to the subject over a first time period that ranges from about 12 to about 18 days prior to harvesting the ovum from the subject. In some embodiments, a donor protocol involves administering a reproductive steroid hormone regimen (e.g., a progesterone regimen) to the subject for a time period of 12, 13, 14, 15, 16, 17, or 18 days prior to harvesting the ovum from the subject. In one preferred embodiment, a donor protocol involves administering a reproductive steroid hormone regimen (e.g., a progesterone regimen) to the subject for a time period of 16-18 days prior to harvesting the ovum from the subject. In one preferred embodiment, a donor protocol involves administering a reproductive steroid hormone regimen (e.g., a progesterone regimen) to the subject for a time period of 17 days prior to harvesting the ovum from the subject.

In some embodiments, a reproductive steroid hormone regimen (e.g., a progesterone regimen) that is administered to the subject during a donor protocol comprises a daily dose of a reproductive steroid hormone (e.g., progesterone), which is administered to the subject on a daily basis for the duration of the first time period. In some embodiments, the daily dose of progesterone from about 150 mg to about 600 mg, which is administered to the subject on a daily basis for the duration of the first time period. In some embodiments, a progesterone regimen that is administered to the subject during a donor protocol comprises a daily dose of 150, 175, 200, 225, 250, 275, 300, 325, 350, 375, 400, 425, 450, 475, 500, 525, 550, 575, or 600 mg of progesterone, which is administered to the subject on a daily basis for the duration of the first time period. In one preferred embodiment, a progesterone regimen that is administered to the subject during a donor protocol comprises a daily dose of 200 to 300 mg of progesterone, which is administered to the subject on a daily basis for the duration of the first time period. In one preferred embodiment, a progesterone regimen that is administered to the subject during a donor protocol comprises a daily dose of 228 mg of progesterone, which is administered to the subject on a daily basis for the duration of the first time period.

In some embodiments, a reproductive steroid hormone regimen that is administered to the subject during a donor protocol comprises a continuous dose of progesterone, which is administered to the subject for the duration of the first time period. In some embodiments, the continuous dose of progesterone is administered to the subject via an implantable device, such as, e.g., a CIDR intravaginal device, which is implanted into the vagina of the subject and left in place for the duration of the first time period. In some embodiments, the continuous dose of progesterone comprises 150, 175, 200, 225, 250, 275, 300, 325, 350, 375, 400, 425, 450, 475 or 500 mg of progesterone or estradiol. In one preferred embodiment, a continuous dose of progesterone comprises 300 mg of progesterone. In one preferred embodiment, a continuous dose of 300 mg of progesterone is administered to the subject via an implantable CIDR intravaginal device that is implanted in the subject's vagina and left in place for the duration of the first time period (e.g., 12-18 days, 16-18 days, 17 days, etc.).

Aspects of the invention include methods that involve administering an estradiol regimen to the subject. In some embodiments, an estradiol regimen comprises a single dose of estradiol. In some embodiments, an estradiol regimen comprises a single dose of estradiol that is administered 6 to 12 days (144 to 288 hours) prior to harvesting an ovum from the subject. In some embodiments, an estradiol regimen comprises a single dose of estradiol that is administered 228 hours to 204 hours prior to harvesting an ovum from the subject. In some embodiments, an estradiol regimen comprises a single dose of estradiol that is administered 216 hours prior to harvesting an ovum from the subject.

In some embodiments, an estradiol regimen comprises a dose of estradiol that ranges from about 100 μg to about 400 μg, such as 105, 110, 115, 120, 125, 130, 135, 140, 145, 150, 155, 160, 165, 170, 175, 180, 185, 190, 195, 200, 205, 210, 215, 216, 217, 218, 219, 220, 221, 222, 223, 224, 225, 226, 227, 228, 229, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, or 395 μg. In one preferred embodiment, an estradiol regimen comprises a single dose of 250 μg of estradiol.

Aspects of the invention include methods that involve administering a releasing hormone regimen to the subject. In some embodiments, a releasing hormone regimen comprises a single dose of a releasing hormone. In some embodiments, a releasing hormone regimen comprises a single dose of a releasing hormone that is administered 4 to 10 days (96 to 240 hours) prior to harvesting an ovum from the subject. In some embodiments, a releasing hormone regimen comprises a single dose of a releasing hormone that is administered 180 hours to 156 hours prior to harvesting an ovum from the subject. In some embodiments, a releasing hormone regimen comprises a single dose of a releasing hormone that is administered 168 hours prior to harvesting an ovum from the subject.

In some embodiments, a releasing hormone regimen comprises a Gonadotropin Releasing Hormone (GnRH) regimen. In some embodiments, a GnRH regimen comprises a dose of GnRH that ranges from about 25 μg to about 500 μg. In some embodiments, a GnRH regimen comprises a dose of GnRH that ranges from about 50 μg to about 200 μg, such as 55, 60, 65, 70, 75, 78, 85, 90, 95, 100, 105, 110, 115, 120, 125, 130, 135, 140, 145, 150, 155, 160, 165, 170, 175, 180, 185, 190, or 195 μg. In one preferred embodiment, a GnRH regimen comprises a single dose of 100 μg of GnRH.

In some embodiments, a releasing hormone regimen comprises a single dose of GnRH that is administered 4 to 10 days (96 to 240 hours) prior to harvesting an ovum from the subject. In some embodiments, a releasing hormone regimen comprises a single dose of GnRH that is administered 180 hours to 156 hours prior to harvesting an ovum from the subject. In some embodiments, a releasing hormone regimen comprises a single dose of GnRH that is administered 168 hours prior to harvesting an ovum from the subject.

Aspects of the invention include methods that involve administering one or more gonadotropin regimens to a subject prior to harvesting an ovum from the subject. In some embodiments, the methods involve administering one or more gonadotropin regimens to a subject over a specified time period prior to harvesting an ovum from the subject. In some embodiments, the time period during which the gonadotropin regimen is administered to the subject is different from the time period during which the progesterone regimen is administered to the subject. In some embodiments, the time period during which the gonadotropin regimen is administered to the subject is the same as the time period during which the progesterone regimen is administered to the subject. In some embodiments, the time period during which the gonadotropin regimen is administered to the subject overlaps with the time period during which the progesterone regimen is administered to the subject.

In some embodiments, a donor protocol involves administering a gonadotropin regimen to the subject over a time period that ranges from about 24 to about 240 hours prior to harvesting the ovum from the subject. In some embodiments, a donor protocol involves administering a gonadotropin regimen to the subject for a time period of about 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238 or 240 hours prior to harvesting the ovum from the subject. In one preferred embodiment, a donor protocol involves administering a gonadotropin regimen to the subject for a time period of 120 to 24 hours prior to harvesting the ovum from the subject. In one preferred embodiment, a donor protocol involves administering a gonadotropin regimen to the subject for a time period of 120 to 36 hours prior to harvesting the ovum from the subject. In one preferred embodiment, a donor protocol comprises a gonadotropin regimen comprising LH, FSH, or a combination thereof.

In some embodiments, a gonadotropin regimen that is administered to the subject during a donor protocol comprises an FSH regimen. In some embodiments, an FSH regimen is administered to a subject over a time period that ranges from about 120 to about 24 hours prior to harvesting an ovum from the subject. In some embodiments, an FSH regimen is administered to a subject over a time period that ranges from about 120, 118, 116, 114, 112, 110, 108, 106, 104, 102, 100, 98, 96, 94, 92, 90, 88, 86, 84, 82, 80, 78, 76, 74, 72, 70, 68, 66, 64, 62, 60, 58, 56, 54, 52, 50, 48, 46, 44, 42, 40, 38, 36, 34, 32, 30, 28, 26, or 24 hours prior to harvesting an ovum from the subject. In one preferred embodiment, an FSH regimen is administered to a subject over a time period that ranges from 120 to 36 hours prior to harvesting an ovum from the subject. In one preferred embodiment, an FSH regimen is administered to a subject over a time period that ranges from 120 to 24 hours prior to harvesting an ovum from the subject.

In some embodiments, an FSH regimen comprises a dose of FSH that ranges from about 100 μg to about 250 μg, such as 105, 110, 115, 120, 125, 130, 135, 140, 145, 150, 155, 160, 165, 170, 175, 180, 185, 190, 195, 200, 205, 210, 215, 220, 225, 230, 235, 240 or 245 μg. In one preferred embodiment, an FSH regimen comprises a single dose of 200 μg of FSH. In one preferred embodiment, an FSH regimen comprises a dose of 200 μg of FSH, which is administered continuously to the subject over the time period of the FSH regimen.

In some embodiments, an FSH regimen comprises a continuous dose of FSH that is administered to a subject over the time period of the FSH regimen. In some embodiments, an FSH regimen comprises a twice daily dose of FSH, wherein doses of FSH are administered 10 to 14 hours apart over the time period of the FSH regimen. In some embodiments, an FSH regimen comprises a diminishing dose of FSH, wherein at the beginning of the dosing regimen a first dose of FSH is administered to the subject, and at one or more subsequent administration time points, the dose of FSH is reduced. In such embodiments, the FSH regimen is administered in a decreasing manner. For example, in one embodiment, an FSH regimen comprises twice daily dosing of FSH, wherein the doses are administered 10 to 14 hours apart over the time period of the FSH regimen, and wherein the FSH doses are as follows: first dose: 167-18.75% of total dosage: second dose 16.7-18.75% of total dosage; third dose: 146-15.6% of total dosage; fourth dose: 14.6-15.6% of total dosage: fifth dose; 9.34-10.42% of total dosage: sixth dose: 9.34-10.42% of total dosage; seventh dose: 6.25-8.33% of total dosage; eighth dose: 6.25-8.33% of total dosage. In some embodiments, an FSH regimen comprises a continuous dose of FSH that is administered continuously for the duration of the time period of the FSH regimen.

In some embodiments, a gonadotropin regimen that is administered to the subject during a donor protocol comprises an LH regimen. In some embodiments, an LH regimen comprises a single dose of LH that is administered to the subject at a time point that ranges from about 5 to about 8 days (120 to 192 hours) prior to harvesting an ovum from the subject. In some embodiments, an LH regimen comprises a single dose of LH that is administered 180 hours to 156 hours prior to harvesting an ovum from the subject. In one preferred embodiment, an LH regimen comprises a single dose of LH that is administered 168 hours prior to harvesting an ovum from the subject.

In some embodiments, an LH regimen comprises a dose of LH that ranges from about 100 μg to about 250 μg, such as 105, 110, 115, 120, 125, 130, 135, 140, 145, 150, 155, 160, 165, 170, 175, 180, 185, 190, 195, 200, 205, 210, 215, 220, 225, 230, 235, 240 or 245 μg. In one preferred embodiment, an LH regimen comprises a single dose of 200 μg of LH. In one preferred embodiment, an LH regimen comprises a dose of 200 μg of LH, which is administered continuously to the subject over the time period of the LH regimen.

In some embodiments, a gonadotropin regimen that is administered to the subject during a donor protocol comprises an hCG regimen. In some embodiments, an hCG regimen comprises a single dose of hCG that is administered to the subject at a time point that ranges from about 5 to about 8 days (120 to 192 hours) prior to harvesting an ovum from the subject. In some embodiments, an hCG regimen comprises a single dose of hCG that is administered 180 hours to 156 hours prior to harvesting an ovum from the subject. In one preferred embodiment, an hCG regimen comprises a single dose of hCG that is administered 168 hours prior to harvesting an ovum from the subject.

In some embodiments, an hCG regimen comprises a single dose of hCG that is administered to the subject 4 to 10 days (96 to 240 hours) prior to harvesting an ovum from the subject. In some embodiments, an hCG regimen comprises a single dose of hCG that is administered 180 hours to 156 hours prior to harvesting an ovum from the subject. In one preferred embodiment, an hCG regimen comprises a single dose of hCG that is administered 168 hours prior to harvesting an ovum from the subject.

In some embodiments, an hCG regimen comprises a dose of hCG that ranges from about 1,750 IU to about 4,000 IU, such as 1,800, 1,850, 1,900, 1,950, 2,000, 2,050, 2,100, 2,150, 2,200, 2,250, 2,300, 2,350, 2,400, 2,450, 2,500, 2,550, 2,600, 2,650, 2,700, 2,750, 2,800, 2,850, 2,900, 2,950, 3,000, 3,050, 3,100, 3,150, 3,200, 3,250, 3,300, 3,350, 3,400, 3,450, 3,500, 3,550, 3,600, 3,650, 3,700, 3,750, 3,800, 3,850, 3,900 or 3,950 IU. In one preferred embodiment, an hCG regimen comprises a single dose of 2,500 IU of hCG.

Aspects of the invention include a donor protocol that comprises administering a prostaglandin regimen to a subject. In some embodiments, a donor protocol involves administering a single dose of prostaglandin to a subject at a time point that ranges from 4 to 6 days prior to harvesting the ovum from the subject. In one preferred embodiment, a donor protocol involves administering a single dose of prostaglandin to a subject 5 days prior to harvesting the ovum from the subject.

In some embodiments, the single dose of prostaglandin ranges from about 100 μg to about 25,000 μg (0.1 to 25 mg), depending on the specific prostaglandin composition that is administered. For example, in some embodiments, a prostaglandin regimen comprises Cloprostenol at a dose that ranges from about 125 μg up to about 625 μg, such as 150, 175, 200, 225, 250, 275, 300, 325, 350, 375, 400, 425, 450, 475, 500, 525, 550, 575 or 600 μg. In one preferred embodiment, a prostaglandin regimen comprises a single dose of 375 μg of Cloprostenol.

In some embodiments, a prostaglandin regimen comprises Dinoprost at a dose that ranges from about 7.5 mg up to about 20 mg, such as 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19 or 19.5 mg. In one preferred embodiment, a prostaglandin regimen comprises a single dose of 15 mg of Dinoprost.

In addition to the dosing regimens described above, aspects of the donor protocols described herein include additional steps relating to, e.g., harvesting an ovum from an animal and conducting in vitro fertilization of the harvested ovum to create an embryo. Any suitable technique(s) known in the art can be used to accomplish ovum harvesting and fertilization, in accordance with knowledge of animal husbandry. In some embodiments, an in vitro fertilized embryo can be frozen prior to implantation. In some embodiments, an in vitro fertilized embryo is not frozen (remains fresh) prior to implantation.

Additionally, aspects of the donor protocols described here can include, in some embodiments, a step of terminating a progesterone regimen. This can be accomplished by ceasing administration of progesterone, and/or removing an implant (e.g., a CIDR intravaginal device) from an animal to cease exposure to progesterone.

As noted above, some small ruminant animals, such as sheep and goats, have seasonal breeding cycles, with most only engaging in breeding activities at certain times of year. The donor protocols described herein can be used for reproductive management of a subject (e.g., a sheep or a goat), either during or outside of a normal breeding season of the subject, thereby maximizing breeding efficiency. Furthermore, the methods described herein can be conducted without having to detect estrus in a subject. This reduces the time and expense involved with reproductive management, especially for animals in whom estrus can be difficult to detect.

In one preferred embodiment, a donor protocol comprises administering a continuous dose of progesterone to a subject for 17 days prior to harvesting an ovum from the subject, wherein the continuous dose of progesterone is administered via a controlled internal drug release (CIDR) intravaginal device comprising 300 mg of progesterone, administering a releasing hormone regimen to the subject, comprising a single dose of 100 μg of gonadotropin releasing hormone (GnRH) administered 168 hours prior to harvesting the ovum from the subject, and administering a gonadotropin hormone regimen to the subject, comprising a continuous dose of 160 to 240 μg of follicle stimulating hormone (FSH) administered over a time period ranging from 120 to 24 hours prior to harvesting the ovum from the subject, and administering a single dose of prostaglandin to the subject 5 days prior to harvesting the ovum from the subject, wherein the single dose of prostaglandin is selected from the group consisting of: 375 μg of Cloprostenol and 15 mg of Dinoprost.

In one preferred embodiment, a donor protocol comprises administering a continuous dose of progesterone to a subject for 17 days prior to harvesting an ovum from the subject, wherein the continuous dose of progesterone is administered via a controlled internal drug release (CIDR) intravaginal device comprising 300 mg of progesterone, administering a releasing hormone regimen to the subject, comprising a single dose of 100 μg of gonadotropin releasing hormone (GnRH) administered 168 hours prior to harvesting the ovum from the subject, and administering a gonadotropin hormone regimen to the subject, comprising a continuous dose of 200 μg of follicle stimulating hormone (FSH) administered over a time period ranging from 120 to 24 hours prior to harvesting the ovum from the subject, and administering a single dose of prostaglandin to the subject 5 days prior to harvesting the ovum from the subject, wherein the single dose of prostaglandin is selected from the group consisting of: 375 μg of Cloprostenol and 15 mg of Dinoprost.

In one preferred embodiment, the subject is a sheep or a goat.

### Recipient Protocol

Aspects of the invention include recipient protocols (methods) that can be used to synchronize ovulation in a plurality of subjects. In some embodiments, a recipient protocol involves administering a reproductive steroid hormone regimen (e.g., a progesterone regimen or an estradiol regimen) and a gonadotropin regimen to the subjects for a period of time prior to achieve synchronization of ovulation in the subjects.

Aspects of the invention include methods that involve administering one or more dosing regimens to a subject over a specified time period prior to achieving synchronization of ovulation in the subjects. For example, in one embodiment, a recipient protocol involves administering a reproductive steroid hormone regimen (e.g., progesterone regimen or an estradiol regimen) to the subjects over a first time period that ranges from about 12 to about 18 days prior to achieving synchronization of ovulation in the subjects. In some embodiments, a recipient protocol involves administering a reproductive steroid hormone regimen (e.g., progesterone regimen) to the subjects for a time period of 12, 13, 14, 15, 16, 17, or 18 days prior to achieving synchronization of ovulation in the subjects. In one preferred embodiment, a recipient protocol involves administering a reproductive steroid hormone regimen (e.g., progesterone regimen) to the subjects for a time period of 16-18 days prior to achieving synchronization of ovulation in the subjects. In one preferred embodiment, a recipient protocol involves administering a reproductive steroid hormone regimen (e.g., progesterone regimen to the subjects for a time period of 17 days prior to achieving synchronization of ovulation in the subjects.

In some embodiments, a reproductive steroid hormone regimen (e.g., progesterone regimen) that is administered to the subjects during a recipient protocol comprises a daily dose of a reproductive steroid hormone (e.g., progesterone), which is administered to the subjects on a daily basis for the duration of the first time period. In some embodiments, the daily dose of progesterone ranges from about 150 mg to about 600 mg, which is administered to the subjects on a daily basis for the duration of the first time period. In some embodiments, a reproductive steroid hormone regimen (e.g., progesterone regimen) that is administered to the subjects during a recipient protocol comprises a daily dose of 150, 175, 200, 225, 250, 275, 300, 325, 350, 375, 400, 425, 450, 475, 500, 525, 550, 575, or 600 mg of progesterone, which is administered to the subjects on a daily basis for the duration of the first time period. In one preferred embodiment, a progesterone regimen that is administered to the subjects during a recipient protocol comprises a daily dose of 200 to 300 mg of progesterone, which is administered to the subjects on a daily basis for the duration of the first time period. In one preferred embodiment, a progesterone regimen that is administered to the subjects during a recipient protocol comprises a daily dose of 228 mg of progesterone, which is administered to the subjects on a daily basis for the duration of the first time period.

In some embodiments, a reproductive steroid hormone regimen that is administered to the subjects during a recipient protocol comprises a continuous dose of progesterone, which is administered to the subjects for the duration of the first time period. In some embodiments, the continuous dose of progesterone is administered to the subjects via an implantable device, such as, e.g., a CIDR intravaginal device, which is implanted into the vaginas of the subjects and left in place for the duration of the first time period. In some embodiments, the continuous dose of progesterone comprises 150, 175, 200, 225, 250, 275, 300, 325, 350, 375, 400, 425, 450, 475 or 500 mg of progesterone or estradiol. In one preferred embodiment, the continuous dose of progesterone comprises 300 mg of progesterone. In one preferred embodiment, a continuous dose of 300 mg of progesterone is administered to the subjects via an implantable CIDR intravaginal device that is implanted in the subjects' vaginas and left in place for the duration of the first time period (e.g., 12-18 days, 16-18 days, 17 days, etc.).

Aspects of the invention include methods that involve administering an estradiol regimen to the subject. In some embodiments, an estradiol regimen comprises a single dose of estradiol. In some embodiments, an estradiol regimen comprises a single dose of estradiol that is administered 1 to 3 days (24 to 72 hours) prior to harvesting an ovum from the subject. In some embodiments, an estradiol regimen comprises a single dose of estradiol that is administered 36 hours to 12 hours prior to harvesting an ovum from the subject. In some embodiments, an estradiol regimen comprises a single dose of estradiol that is administered 24 hours prior to harvesting an ovum from the subject.

In some embodiments, an estradiol regimen comprises a dose of estradiol that ranges from about 100 μg to about 400 μg, such as 105, 110, 115, 120, 125, 130, 135, 140, 145, 150, 155, 160, 165, 170, 175, 180, 185, 190, 195, 200, 205, 210, 215, 216, 217, 218, 219, 220, 221, 222, 223, 224, 225, 226, 227, 228, 229, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, or 395 μg. In one preferred embodiment, an estradiol regimen comprises a single dose of 250 μg of estradiol.

Aspects of the invention include methods that involve administering one or more gonadotropin regimens to a plurality of subjects prior to achieving synchronization of ovulation in the subjects. In some embodiments, a gonadotropin regimen that is administered to the subjects during a recipient protocol comprises an hCG regimen. In some embodiments, an hCG regimen comprises a single dose of hCG that is administered to the subjects at a time point that ranges from about 12 to about 36 hours prior to achieving synchronization of ovulation in the subjects. In some embodiments, an hCG regimen comprises a single dose of hCG that is administered 24 hours prior to achieving synchronization of ovulation in the subjects.

In some embodiments, an hCG regimen comprises a dose of hCG that ranges from about 50 IU to about 200 IU, such as 55, 60, 65, 70, 75, 80, 85, 90, 95, 100, 105, 110, 115, 120, 125, 130, 135, 140, 145, 150, 155, 160, 165, 170, 175, 180, 185, 190 or 195 IU. In one preferred embodiment, an hCG regimen comprises a single dose of 100 IU of hCG.

In some embodiments, an hCG regimen comprises a dose of hCG that ranges from about 1,750 IU to about 4,000 IU, such as 1,800, 1,850, 1,900, 1,950, 2,000, 2,050, 2,100, 2,150, 2,200, 2,250, 2,300, 2,350, 2,400, 2,450, 2,500, 2,550, 2,600, 2,650, 2,700, 2,750, 2,800, 2,850, 2,900, 2,950, 3,000, 3,050, 3,100, 3,150, 3,200, 3,250, 3,300, 3,350, 3,400, 3,450, 3,500, 3,550, 3,600, 3,650, 3,700, 3,750, 3,800, 3,850, 3,900 or 3,950 IU. In one preferred embodiment, an hCG regimen comprises a single dose of 2,500 IU of hCG.

In some embodiments, a gonadotropin regimen that is administered to the subjects during a recipient protocol comprises an eCG regimen. In some embodiments, an eCG regimen comprises a single dose of eCG that is administered to the subjects at a time point that ranges from about 12 to about 36 hours prior to achieving synchronization of ovulation in the subjects. In some embodiments, an eCG regimen comprises a single dose of eCG that is administered 24 hours prior to achieving synchronization of ovulation in the subjects.

In some embodiments, an eCG regimen comprises a dose of eCG that ranges from about 100 IU to about 400 IU, such as 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390 or 395 IU. In one preferred embodiment, an eCG regimen comprises a single dose of 200 IU of eCG.

In some embodiments, a gonadotropin regimen that is administered to the subjects during a recipient protocol comprises an eCG regimen and an hCG regimen. In some embodiments, a gonadotropin regimen comprises a single dose of eCG that ranges from 100 to 400 IU, and a single dose of hCG that ranges from 50 to 200 IU. In some preferred embodiment, a gonadotropin regimen comprises a single dose of 200 IU of eCG, and a single dose of 100 IU of hCG. In some embodiments, a gonadotropin regimen comprising a single dose of hCG and a single dose of eCG is administered to the subjects at a time point that ranges from about 12 to about 36 hours prior to achieving synchronization of ovulation in the subjects. In some embodiments, a gonadotropin regimen comprising a single dose of hCG and a single dose of eCG is administered to the subjects 24 hours prior to achieving synchronization of ovulation in the subjects.

Aspects of the invention include a recipient protocol that comprises administering a prostaglandin regimen to a plurality of subjects. In some embodiments, a recipient protocol involves administering a single dose of prostaglandin to the subjects at a time point that ranges from 12 to 36 hours prior to achieving synchronization of ovulation in the subjects. In one preferred embodiment, a recipient protocol involves administering a single dose of prostaglandin to the subjects 24 hours prior to achieving synchronization of ovulation in the subjects.

In some embodiments, the single dose of prostaglandin ranges from about 100 μg to about 25,000 μg (0.1 to 25 mg), depending on the specific prostaglandin composition that is administered. For example, in some embodiments, a prostaglandin regimen comprises Cloprostenol at a dose that ranges from about 125 μg up to about 625 μg, such as 150, 175, 200, 225, 250, 275, 300, 325, 350, 375, 400, 425, 450, 475, 500, 525, 550, 575 or 600 μg. In one preferred embodiment, a prostaglandin regimen comprises a single dose of 375 μg of Cloprostenol.

In some embodiments, a prostaglandin regimen comprises Dinoprost at a dose that ranges from about 7.5 mg up to about 20 mg, such as 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19 or 19.5 mg. In one preferred embodiment, a prostaglandin regimen comprises a single dose of 15 mg of Dinoprost.

In addition to the dosing regimens described above, aspects of the recipient protocols described herein include additional steps relating to, e.g., verifying ovulation in one or more of the plurality of subjects. In some embodiments, verifying ovulation comprises detecting a corpus luteum in an ovary of one or more of the subjects, which can be accomplished using techniques known in the art, in accordance with knowledge of animal husbandry. In addition, aspects of the recipient protocols described herein include additional steps relating to, e.g,, transferring an embryo into a recipient animal in order to establish a pregnancy in the animal. Any suitable technique(s) known in the art can be used to accomplish embryo transfer, in accordance with knowledge of animal husbandry. In some embodiments, an in vitro fertilized embryo can be frozen prior to implantation. In some embodiments, an in vitro fertilized embryo is not frozen (remains fresh) prior to implantation.

Additionally, aspects of the recipient protocols described here can include, in some embodiments, a step of terminating a reproductive steroid hormone regimen (e.g., a progesterone regimen or an estradiol regimen). This can be accomplished by ceasing administration of the reproductive steroid hormone, and/or removing an implant (e.g., a CIDR intravaginal device) from an animal to cease exposure to the reproductive steroid hormone. In some embodiments, a recipient protocol comprises terminating a reproductive steroid hormone regimen at the time of synchronization of ovulation. In some embodiments, a recipient protocol comprises terminating a reproductive steroid hormone regimen at the time that the prostaglandin regimen is administered. In some embodiments, a recipient protocol comprises terminating a reproductive steroid hormone regimen at the time that the gonadotropin regimen is administered.

As noted above, some small ruminant animals, such as sheep and goats, have seasonal breeding cycles, with most only engaging in breeding activities at certain times of year. The recipient protocols described herein can be used for reproductive management of a subject (e.g., a sheep or a goat), either during or outside of a normal breeding season of the subject, thereby maximizing breeding efficiency. Furthermore, the methods described herein can be conducted without having to detect estrus in a subject. This reduces the time and expense involved with reproductive management, especially for animals in whom estrus can be difficult to detect. In some embodiments, estrus detection is conducted in order to verify that the recipient protocol achieved the desired outcome.

In one preferred embodiment, a recipient protocol comprises administering a continuous dose of progesterone to the subjects for 17 days prior to synchronization of ovulation, wherein the continuous dose of progesterone is administered via a controlled internal drug release (CIDR) intravaginal device comprising 300 mg of progesterone; administering a gonadotropin regimen to the subjects 24 hours prior to synchronization of ovulation, comprising a single dose of 200 IU of eCG, and administering a single dose of prostaglandin to the subjects 24 hours prior to synchronization of ovulation, wherein the single dose of prostaglandin is selected from the group consisting of: 375 μg of Cloprostenol and 15 mg of Dinoprost.

In one preferred embodiment, a recipient protocol comprises administering a continuous dose of progesterone to the subjects for 17 days prior to synchronization of ovulation, wherein the continuous dose of progesterone is administered via a controlled internal drug release (CIDR) intravaginal device comprising 300 mg of progesterone; administering a gonadotropin regimen to the subjects 24 hours prior to synchronization of ovulation, comprising a single dose of 100 IU of hCG, and administering a single dose of prostaglandin to the subjects 24 hours prior to synchronization of ovulation, wherein the single dose of prostaglandin is selected from the group consisting of: 375 μg of Cloprostenol and 15 mg of Dinoprost.

In one preferred embodiment, a recipient protocol comprises administering a continuous dose of progesterone to the subjects for 17 days prior to synchronization of ovulation, wherein the continuous dose of progesterone is administered via a controlled internal drug release (CIDR) intravaginal device comprising 300 mg of progesterone; administering a gonadotropin regimen to the subjects 24 hours prior to synchronization of ovulation, comprising a single dose of 200 IU of eCG and a single dose of 100 IU of hCG, and administering a single dose of prostaglandin to the subjects 24 hours prior to synchronization of ovulation, wherein the single dose of prostaglandin is selected from the group consisting of: 375 μg of Cloprostenol and 15 mg of Dinoprost.

In one preferred embodiment, the subject is a sheep or a goat.

### Pregnancy Protocol

Aspects of the invention include pregnancy protocols (methods) that combine aspects of the donor and recipient protocols described herein to establish a pregnancy in one or more subjects (e.g., one or more sheep or goats). Any of the donor protocols described herein can be combined with any of the recipient protocols described herein in order to establish a pregnancy in one or more subjects. In some embodiments, an in vitro fertilized embryo can be frozen prior to implantation. In some embodiments, an in vitro fertilized embryo is not frozen (remains fresh) prior to implantation.

In one preferred embodiment, a pregnancy protocol comprises administering a continuous dose of progesterone to a donor animal for 17 days prior to harvesting an ovum from the donor animal. wherein the continuous dose of progesterone is administered via a controlled internal drug release (CIDR) intravaginal device comprising 300 mg of progesterone, administering a releasing hormone regimen to the donor animal, comprising a single dose of 100 μg of gonadotropin releasing hormone (GnRH) administered 168 hours prior to harvesting the ovum from the donor animal, and administering a gonadotropin hormone regimen to the donor animal, comprising a continuous dose of 160 to 240 μg of follicle stimulating hormone (FSH) administered over a time period ranging from 120 to 24 hours prior to harvesting the ovum from the donor animal, and administering a single dose of prostaglandin to the donor animal 5 days prior to harvesting the ovum from the donor animal, wherein the single dose of prostaglandin is selected from the group consisting of: 375 μg of Cloprostenol and 15 mg of Dinoprost, administering a continuous dose of progesterone to the recipient animal for 17 days prior to synchronization of ovulation, wherein the continuous dose of progesterone is administered via a controlled internal drug release (CIDR) intravaginal device comprising 300 mg of progesterone; administering a gonadotropin regimen to the recipient animal 24 hours prior to synchronization of ovulation, comprising a single dose of 200 IU of eCG, and administering a single dose of prostaglandin to the recipient animal 24 hours prior to synchronization of ovulation, wherein the single dose of prostaglandin is selected from the group consisting of: 375 μg of Cloprostenol and 15 mg of Dinoprost, harvesting an ovum from the donor animal, fertilizing the ovum to generate an embryo, and transferring the fertilized embryo into the recipient animal to establish a pregnancy. In one preferred embodiment, the gonadotropin hormone regimen comprises a continuous dose of 200 μg of FSH.

In one preferred embodiment, a pregnancy protocol comprises administering a continuous dose of progesterone to a donor animal for 17 days prior to harvesting an ovum from the donor animal. wherein the continuous dose of progesterone is administered via a controlled internal drug release (CIDR) intravaginal device comprising 300 mg of progesterone, administering a releasing hormone regimen to the donor animal, comprising a single dose of 100 μg of gonadotropin releasing hormone (GnRH) administered 168 hours prior to harvesting the ovum from the donor animal, and administering a gonadotropin hormone regimen to the donor animal, comprising a continuous dose of 160 to 240 μg of follicle stimulating hormone (FSH) administered over a time period ranging from 120 to 24 hours prior to harvesting the ovum from the donor animal, and administering a single dose of prostaglandin to the donor animal 5 days prior to harvesting the ovum from the donor animal, wherein the single dose of prostaglandin is selected from the group consisting of: 375 μg of Cloprostenol and 15 mg of Dinoprost, administering a continuous dose of progesterone to the recipient animal for 17 days prior to synchronization of ovulation, wherein the continuous dose of progesterone is administered via a controlled internal drug release (CIDR) intravaginal device comprising 300 mg of progesterone; administering a gonadotropin regimen to the recipient animal 24 hours prior to synchronization of ovulation, comprising a single dose of 100 IU of hCG, and administering a single dose of prostaglandin to the recipient animal 24 hours prior to synchronization of ovulation, wherein the single dose of prostaglandin is selected from the group consisting of: 375 μg of Cloprostenol and 15 mg of Dinoprost. harvesting an ovum from the donor animal, fertilizing the ovum to generate an embryo, and transferring the fertilized embryo into the recipient animal to establish a pregnancy. In one preferred embodiment, the gonadotropin hormone regimen comprises a continuous dose of 200 μg of FSH.

In one preferred embodiment, a pregnancy protocol comprises administering a continuous dose of progesterone to a donor animal for 17 days prior to harvesting an ovum from the donor animal. wherein the continuous dose of progesterone is administered via a controlled internal drug release (CIDR) intravaginal device comprising 300 mg of progesterone, administering a releasing hormone regimen to the donor animal, comprising a single dose of 100 μg of gonadotropin releasing hormone (GnRH) administered 168 hours prior to harvesting the ovum from the donor animal, and administering a gonadotropin hormone regimen to the donor animal, comprising a continuous dose of 160 to 240 μg of follicle stimulating hormone (FSH) administered over a time period ranging from 120 to 24 hours prior to harvesting the ovum from the donor animal, and administering a single dose of prostaglandin to the donor animal 5 days prior to harvesting the ovum from the donor animal, wherein the single dose of prostaglandin is selected from the group consisting of: 375 μg of Cloprostenol and 15 mg of Dinoprost, administering a continuous dose of progesterone to the recipient animal for 17 days prior to synchronization of ovulation, wherein the continuous dose of progesterone is administered via a controlled internal drug release (CIDR) intravaginal device comprising 300 mg of progesterone; administering a gonadotropin regimen to the recipient animal 24 hours prior to synchronization of ovulation, comprising a single dose of 100 IU of hCG and a single dose of 200 IU of cCG, and administering a single dose of prostaglandin to the recipient animal 24 hours prior to synchronization of ovulation, wherein the single dose of prostaglandin is selected from the group consisting of: 375 μg of Cloprostenol and 15 mg of Dinoprost, harvesting an ovum from the donor animal. fertilizing the ovum to generate an embryo, and transferring the fertilized embryo into the recipient animal to establish a pregnancy. In one preferred embodiment, the gonadotropin hormone regimen comprises a continuous dose of 200 μg of FSH.

As noted above, some small ruminant animals, such as sheep and goats, have seasonal breeding cycles, with most only engaging in breeding activities at certain times of year. The pregnancy protocols described herein can be used for reproductive management of a subject (e.g., a sheep or a goat), either during or outside of a normal breeding season of the subject, thereby maximizing breeding efficiency. Furthermore, the methods described herein can be conducted without having to detect estrus in a subject. This reduces the time and expense involved with reproductive management, especially for animals in whom estrus can be difficult to detect. In some embodiments, estrus detection is conducted in order to verify that the recipient protocol achieved the desired outcome. Aspects of the invention include successfully achieving a pregnancy in a subject at a rate that ranges from about 50 to 100 percent (%), such 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99 percent (%). Pregnancy rate as used herein is calculated by dividing the number of successfully established pregnancies by the number of attempts at establishing a pregnancy in an animal that had undergone a recipient protocol as described herein.

In one preferred embodiment, the subject is a sheep or a goat.

### Kits:

Aspects of the invention include kits comprising the compositions and formulations thereof, of the disclosure and instructions for use. Kits in accordance with embodiments of the invention typically include a label indicating the intended use of the contents of the kit, The term “label” as used herein includes any writing, or recorded material supplied on or with a kit, or which otherwise accompanies a kit.

In some embodiments, a kit comprises a composition packaged in a container. In some embodiments, a kit can further comprise instructions that direct administration of a unit dose of a composition to a subject. In some embodiments, a kit comprises one or more vials, each vial containing a composition for administration to an animal (e.g., a sheep or a goat). In some embodiments, a kit comprises one or more prefilled syringes, each prefilled syringe containing a composition for administration to an animal (e.g., a sheep or a goat). In some embodiments, a kit comprises a vial or a prefilled syringe containing a mixture of two or more compositions to be administered simultaneously to an animal (e.g., a sheep or a goat).

The invention now being fully described, it will be apparent to one of ordinary skill in the art that various changes and modifications can be made without departing from the spirit or scope of the invention.

### EXAMPLES

### Example 1: Comparison of Pregnancy Rates

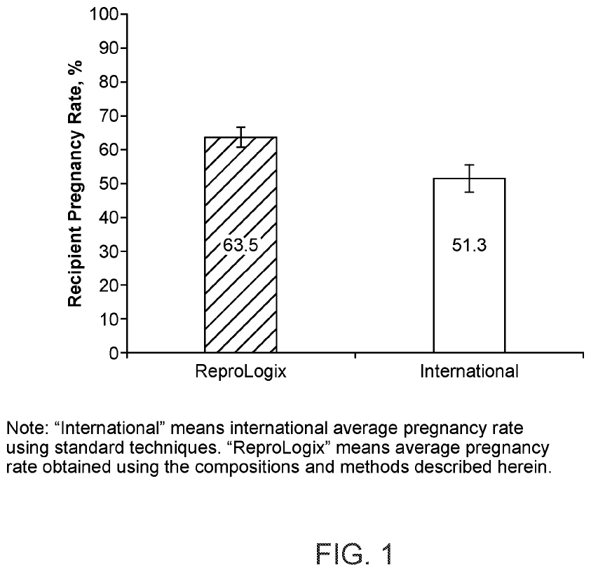

FIG. 1 is a graph showing pregnancy rates for embryo transfer of fresh, small ruminant IVP embryos produced using the protocols described herein (255 recipients, labeled “ReproLogix”) in comparison to internationally collected data (3,612 recipients). Data for the analysis was adapted from: Ptak et al., 2013; Rodríguez-Dorta et al., 2007; Cognié et al., 2004: Cognié et al., 2001: Ptak et al., 1999; Brown and Radziewic, 1998: Silva et al., 2012: Baldassarre et al., 2007: Guo et al., 2008; Loi et al., 2018. A literature review yielded little data for pregnancy data for sheep and goat IVP embryos produced and transferred within the United States.

Data for ReproLogix was compiled through records of pregnancy rates on ReproLogix-owned recipient animals synchronized using the protocols described herein and that underwent embryo transfers performed by the ReproLogix staff. International data was collected through review of current literature concerning embryo transfer of sheep or goat in vitro produced (IVP) embryo. Only data from embryo transfers of fresh in vitro fertilized embryos was included. The results demonstrate that the subject protocols achieved an overall average pregnancy rate of 63.5%, in comparison to the international average pregnancy rate of 51.3%.

### REFERENCES

Baldassarre H, Rao KM, Neveu N, Brochu E, Begin I, Behboodi E, Hockley DK. Laparoscopic ovum pick-up followed by in vitro embryo production for the reproductive rescue of aged goats of high genetic value. Reprod Fertil Dev. 2007; 19 (5): 612-6, doi: 10,1071/rd07024, PMID: 17601408.

Brown BW, Radziewic T. Production of sheep embryos in vitro and development of progeny following single and twin embryo transfers. Theriogenology, 1998 Jun; 49 (8): 1525-36, doi: 10.1016/s0093-691x (98) 00098-3, PMID: 10732016.

Cognié, Yves, et al. “Embryo survival after transfer of in vitro and in vivo produced goat embryos,” Proceedings: 17th Annual Meeting AETE, France, 2001.

Cognié Y, Poulin N, Locatelli Y, Mermillod P. State-of-the-art production, conservation and transfer of in-vitro-produced embryos in small ruminants. Reprod Fertil Dev. 2004: 16(4): 437-45, doi: 10.10371/RD04029, PMID: 15315742.

Gou KM, Guan H, Bai JH, Cui XH, Wu Z F, Yan FX. An XR. Field evaluation of juvenile in vitro embryo transfer (JIVET) in sheep. Anim Reprod Sci. 2009 June: 112 (3-4): 316-24, doi: 10.1016/j.anireprosci.2008.05.008, Epub 2008 May 15, PMID: 18565700.

Loi P, Galli C, Lazzari G, Matsukawa K, Fulka J Jr, Goeritz F, Hildebrandt TB. Development to term of sheep embryos reconstructed after inner cell mass/trophoblast exchange. J Reprod Dev. 2018 Apr. 13; 64 (2):187-191, doi: 10.1262/jrd.2017-109, Epub 2018 Feb. 15, PMID: 29445070; PMCID: PMC5902907.

Papadopoulos, S., Rizos, D., Duffy, P., Wade, M., Quinn, K., Boland, M.P., & Lonergan, P. (2002). Embryo survival and recipient pregnancy rates after transfer of fresh or vitrified, in vivo or in vitro produced ovine blastocysts. Animal reproduction science, 74 (1-2), 35-44.

Ptak G, Dattena M, Loi P, Tischner M, Cappai P. Ovum pick-up in sheep: efficiency of in vitro embryo production, vitrification and birth of offspring. Theriogenology, 1999 Oct, 15: 52 (6): 1105-14, doi: 10.1016/S0093-691X (99) 00198-3, PMID: 10735116.

Ptak GE, D+Agostino A, Toschi P, Fidanza A, Zacchini F, Czernik M, Monaco F, Loi P. Post-implantation mortality of in vitro produced embryos is associated with DNA methyltransferase 1 dysfunction in sheep placenta. Hum Reprod. 2013 Feb.; 28 (2): 298-305, doi: 10.1093/humrep/des397, Epub 2012 Nov. 20, PMID: 23169866.

Rodríguez-Dorta N, CogniéY, González F, Poulin N, Guignot F, Touzé J L, Baril G, Cabrera F, Alamo D, Batista M, Gracia A, Mermillod P. Effect of coculture with oviduct epithelial cells on viability after transfer of vitrified in vitro produced goat embryos, Theriogenology. 2007 Oct. 1; 68(6): 908-13, doi: 10.1016/j.theriogenology.2007.07.004. Epub 2007 Aug. 27, PMID: 17719625,

Silva, J.C.B., W.K. Okabe, and A.S. Traldi. “From cattle to sheep; a view of the difficulties and success of commercial in vitro production of sheep embryos,” Animal Reproduction (AR) 9.3 (2018): 195-200.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of stimulating follicular maturation in a sheep or goat, the method comprising:
administering a reproductive steroid hormone regimen to the sheep or goat prior to harvesting an ovum from the sheep or goat; and
administering a gonadotropin hormone regimen to the sheep or goat prior to harvesting the ovum from the sheep or goat.

2. The method of claim 1, further comprising administering a releasing hormone regimen to the sheep or goat prior to harvesting the ovum from the sheep or goat.

3. The method of claim 1 or 2, wherein the reproductive steroid hormone regimen is administered over a first time period prior to harvesting the ovum from the sheep or goat.

4. The method of claim of any one of claims 1-3, wherein the reproductive steroid hormone regimen comprises a progesterone regimen.

5. The method of claim 4, wherein the progesterone regimen comprises a daily dose of progesterone that is administered to the sheep or goat during the first time period.

6. The method of claim 5, wherein the daily dose of progesterone ranges from 150 mg to 600 mg.

7. The method of claim 6, wherein the daily dose of progesterone ranges from 200 mg to 300 mg.

8. The method of claim 7, wherein the daily dose of progesterone is 228 mg.

9. The method of claim 3, wherein the progesterone regimen comprises a continuous dose of progesterone that is administered to the sheep or goat during the first time period.

10. The method of claim 9, wherein the continuous dose of progesterone comprises a controlled internal drug release (CIDR) intravaginal device.

11. The method of claim 10, wherein the CIDR comprises 150 mg to 450 mg of progesterone.

12. The method of claim 11, wherein the CIDR comprises 300 mg of progesterone.

13. The method of any one of claims 3-12, wherein the first time period ranges from 12 to 18 days prior to harvesting the ovum from the sheep or goat.

14. The method of claim 13, wherein the first time period ranges from 16 to 18 days prior to harvesting the ovum from the sheep or goat.

15. The method of claim 14, wherein the first time period is 17 days prior to harvesting the ovum from the sheep or goat.

16. The method of any one of claims 2-15, wherein the releasing hormone regimen comprises a single dose of a releasing hormone.

17. The method of claim 16, wherein the releasing hormone comprises Gonadotropin Releasing Hormone (GnRH).

18. The method of claim 17, wherein the single dose of GnRH ranges from 50 μg to 200 μg.

19. The method of claim 18, wherein the single dose of GnRH is 100 μg.

20. The method of any one of claims 16-19, wherein the single dose of GnRH is administered to the sheep or goat 180 hours to 156 hours prior to harvesting the ovum from the sheep or goat.

21. The method of claim 20, wherein the single dose of GnRH is administered to the sheep or goat 168 hours prior to harvesting the ovum from the sheep or goat.

22. The method of any one of claims 1-21, wherein the gonadotropin hormone regimen comprises a gonadotropin hormone selected from the group consisting of: follicle stimulating hormone (FSH), luteinizing hormone (LH), human chorionic gonadotropin (hCG), and any combination thereof.

23. The method of claim 22, wherein the gonadotropin hormone regimen comprises FSH.

24. The method of claim 23, wherein the FSH is administered over a second time period prior to harvesting the ovum from the sheep or goat.

25. The method of claim 24, wherein the second time period ranges from 120 to 24 hours prior to harvesting the ovum from the sheep or goat.

26. The method of claim 24, wherein the second time period ranges from 120 to 36 hours prior to harvesting the ovum from the sheep or goat.

27. The method of any one of claims 23-26, wherein the FSH comprises a dose that ranges from 160 μg to 240 μg.

28. The method of claim 27, wherein the FSH dose is 200 μg.

29. The method of any one of claims 23-28, wherein the FSH is administered continuously over the second time period.

30. The method of any one of claims 23-28, wherein the FSH is administered via twice daily doses that are administered 10-14 hours apart over the second time period.

31. The method of claim 30, wherein the twice daily doses are administered in a decreasing manner over the second time period.

32. The method of any one of claims 22-31, wherein the gonadotropin hormone regimen comprises a single dose of LH.

33. The method of claim 32, wherein the single dose of LH ranges from 12 mg to 50 mg.

34. The method of claim 33, wherein the single dose of LH is 25 mg.

35. The method of any one of claims 32-34, wherein the single dose of LH is administered to the sheep or goat 180 hours to 156 hours prior to harvesting the ovum from the sheep or goat.

36. The method of claim 35, wherein the single dose of LH is administered to the sheep or goat 168 hours prior to harvesting the ovum from the sheep or goat.

37. The method of any one of claims 22-31, wherein the gonadotropin hormone regimen comprises a single dose of hCG.

38. The method of claim 37, wherein the single dose of hCG ranges from 50 IU to 200 IU.

39. The method of claim 38, wherein the single dose of hCG is 100 IU.

40. The method of claim 37, wherein the single dose of hCG ranges from 1,750 IU to 4,000 IU.

41. The method of claim 40, wherein the single dose of hCG is 2,500 IU.

42. The method of any one of claims 37-41, wherein the single dose of hCG is administered to the sheep or goat 180 hours to 156 hours prior to harvesting the ovum from the sheep or goat.

43. The method of claim 42, wherein the single dose of hCG is administered to the sheep or goat 168 hours prior to harvesting the ovum from the sheep or goat.

44. The method of any one of claims 1-43, further comprising administering a prostaglandin regimen to the sheep or goat prior to harvesting the ovum from the sheep or goat.

45. The method of claim 44, wherein the prostaglandin regimen comprises a single dose of prostaglandin that is administered 4 to 6 days prior to harvesting the ovum from the sheep or goat.

46. The method of claim 45, wherein the single dose of prostaglandin is administered 5 days prior to harvesting the ovum from the sheep or goat.

47. The method of any one of claims 45-46, wherein the prostaglandin is Cloprostenol or Dinoprost.

48. The method of claim 47, wherein the prostaglandin is Cloprostenol, and the single dose of Cloprostenol ranges from 125 mg to 625 mg.

49. The method of claim 48, wherein the single dose of Cloprostenol is 375 mg.

50. The method of claim 47, wherein the prostaglandin is Dinoprost, and the single dose of Dinoprost ranges from 7.5 mg to 20 mg.

51. The method of claim 50, wherein the single dose of Dinoprost is 15 mg.

52. The method of any one of claims 1-51, further comprising harvesting the ovum from the sheep or goat.

53. The method of any one of claims 1-52, further comprising terminating the progesterone regimen.

54. The method of any one of claims 1-53, further comprising fertilizing the ovum to generate an embryo.

55. The method of any one of claims 1-54, which is conducted during a breeding season of the sheep or goat.

56. The method of any one of claims 1-55, which is conducted outside of a breeding season of the sheep or goat.

57. A method of stimulating follicular maturation in a sheep or goat, the method comprising:
administering a continuous dose of progesterone to the sheep or goat for 17 days prior to harvesting an ovum from the sheep or goat, wherein the continuous dose of progesterone is administered via a controlled internal drug release (CIDR) intravaginal device comprising 300 mg of progesterone;
administering a releasing hormone regimen to the sheep or goat, comprising a single dose of 100 μg of gonadotropin releasing hormone (GnRH) administered 168 hours prior to harvesting the ovum from the sheep or goat; and
administering a gonadotropin hormone regimen to the sheep or goat, comprising a continuous dose of 160 to 240 μg of follicle stimulating hormone (FSH) administered over a time period ranging from 120-24 hours prior to harvesting the ovum from the sheep or goat; and
administering a single dose of prostaglandin to the sheep or goat 5 days prior to harvesting the ovum from the sheep or goat, wherein the single dose of prostaglandin is selected from the group consisting of: 375 μg of Cloprostenol and 15 mg of Dinoprost.

58. A method of generating a fertilized embryo from a sheep or goat, the method comprising:
administering a continuous dose of progesterone to the sheep or goat for 17 days prior to harvesting an ovum from the sheep or goat, wherein the continuous dose of progesterone is administered via a controlled internal drug release (CIDR) intravaginal device comprising 300 mg of progesterone;
administering a releasing hormone regimen to the sheep or goat, comprising a single dose of 100 μg of gonadotropin releasing hormone (GnRH) administered 168 hours prior to harvesting the ovum from the sheep or goat; and
administering a gonadotropin hormone regimen to the sheep or goat, comprising a continuous dose of 160 to 240 μg of follicle stimulating hormone (FSH) administered over a time period ranging from 120-24 hours prior to harvesting the ovum from the sheep or goat;
administering a single dose of prostaglandin to the sheep or goat 5 days prior to harvesting the ovum from the sheep or goat, wherein the single dose of prostaglandin is selected from the group consisting of: 375 μg of Cloprostenol and 15 mg of Dinoprost;
harvesting the ovum from the sheep or goat; and
fertilizing the ovum to generate an embryo.

59. A method of synchronizing ovulation in a plurality of sheep or goats, the method comprising:
administering a reproductive steroid regimen to the sheep or goats; and
administering a gonadotropin hormone regimen to the sheep or goats.

60. The method of claim 59, wherein the reproductive steroid hormone regimen is administered over a first time period prior to ovulation synchronization.

61. The method of claim of any one of claims 59-60, wherein the reproductive steroid hormone regimen comprises a progesterone regimen.

62. The method of claim 61, wherein the progesterone regimen comprises a daily dose of progesterone that is administered to the sheep or goats during the first time period.

63. The method of claim 62, wherein the daily dose of progesterone ranges from 150 mg to 600 mg.

64. The method of claim 63, wherein the daily dose of progesterone ranges from 200 mg to 300 mg.

65. The method of claim 64, wherein the daily dose of progesterone is 228 mg.

66. The method of claim 61, wherein the progesterone regimen comprises a continuous dose of progesterone that is administered to the sheep or goats during the first time period.

67. The method of claim 66, wherein the continuous dose of progesterone comprises a controlled internal drug release (CIDR) intravaginal device.

68. The method of claim 67, wherein the CIDR comprises 150 mg to 450 mg of progesterone.

69. The method of claim 68, wherein the CIDR comprises 300 mg of progesterone.

70. The method of any one of claims 61-69, wherein the first time period ranges from 12 to 18 days prior to ovulation synchronization.

71. The method of claim 70, wherein the first time period ranges from 16 to 18 days prior to ovulation synchronization.

72. The method of claim 71, wherein the first time period is 17 days prior to ovulation synchronization.

73. The method of any one of claims 59-72, wherein the gonadotropin hormone regimen comprises a gonadotropin hormone selected from the group consisting of: equine chorionic gonadotropin (eCG), human chorionic gonadotropin (hCG), and any combination thereof.

74. The method of claim 73, wherein the gonadotropin hormone regimen comprises administering a single dose of eCG to the sheep or goats.

75. The method of claim 74, wherein the single dose of eCG ranges from 100 to 400 IU.

76. The method of claim 75, wherein the single dose of eCG is 200 IU.

77. The method of claim 73, wherein the gonadotropin hormone regimen comprises administering a single dose of hCG to the sheep or goats.

78. The method of claim 77, wherein the single dose of hCG ranges from 50 to 200 IU.

79. The method of claim 78, wherein the single dose of hCG is 100 IU.

80. The method of claim 77, wherein the single dose of hCG ranges from 1,750 IU to 4,000 IU.

81. The method of claim 80, wherein the single dose of hCG is 2,500 IU.

82. The method of claim 73, wherein the gonadotropin hormone regimen comprises a single dose of eCG ranging from 100 to 400 IU and a single dose of hCG ranging from 50 to 200 IU.

83. The method of claim 82, wherein the gonadotropin hormone regimen comprises a single dose of 200 IU of eCG and a single dose of 100 IU of hCG.

84. The method of any one of claims 73-83, wherein the gonadotropin hormone regimen is administered to the sheep or goats 12 to 36 hours prior to ovulation synchronization.

85. The method of claim 84, wherein the gonadotropin hormone regimen is administered to the sheep or goats 24 hours prior to ovulation synchronization.

86. The method of any one of claims 59-85, further comprising administering a prostaglandin regimen to the sheep or goats prior to ovulation synchronization.

87. The method of claim 86, wherein the prostaglandin regimen comprises a single dose of prostaglandin that is administered 12 to 36 hours prior to ovulation synchronization.

88. The method of claim 87, wherein the single dose of prostaglandin is administered 24 hours prior to ovulation synchronization.

89. The method of any one of claims 87-88, wherein the prostaglandin is Cloprostenol or Dinoprost.

90. The method of claim 89, wherein the prostaglandin is Cloprostenol, and the single dose of Cloprostenol ranges from 125 μg to 625 μg.

91. The method of claim 90, wherein the single dose of Cloprostenol is 375 μg.

92. The method of claim 89, wherein the prostaglandin is Dinoprost, and the single dose of Dinoprost ranges from 7.5 mg to 20 mg.

93. The method of claim 92, wherein the single dose of Dinoprost is 15 mg.

94. The method of any one of claims 59-93, resulting in a 90% or greater rate of synchronization of ovulation between the sheep or goats.

95. The method of any one of claims 59-94, further comprising verifying ovulation in one or more of the sheep or goats.

96. The method of claim 95, wherein verifying ovulation comprises detecting a corpus luteum in an ovary of one or more of the sheep or goats.

97. The method of any one of claims 59-96, further comprising detecting estrus in one or more of the sheep or goats.

98. The method of any one of claims 59-97, further comprising transferring an in vitro fertilized embryo into one or more of the sheep or goats.

99. The method of claim 98, wherein the in vitro fertilized embryo is transferred into the sheep or goats 7 to 8 days after ovulation synchronization.

100. The method of any one of claims 59-99, further comprising terminating the progesterone regimen upon ovulation synchronization.

101. A method of synchronizing ovulation in a plurality of sheep or goats, the method comprising:
administering a continuous dose of progesterone to the sheep or goats for 17 days prior to synchronization of ovulation, wherein the continuous dose of progesterone is administered via a controlled internal drug release (CIDR) intravaginal device comprising 300 mg of progesterone;
administering a gonadotropin regimen to the sheep or goats 24 hours prior to synchronization of ovulation, comprising a single dose of 200 IU of eCG and a single dose of 100 IU of hCG; and
administering a single dose of prostaglandin to the sheep or goats 24 hours prior to synchronization of ovulation, wherein the single dose of prostaglandin is selected from the group consisting of: 375 μg of Cloprostenol and 15 mg of Dinoprost.

102. A method of establishing a pregnancy in a sheep or goat, the method comprising:
administering a continuous dose of progesterone to the sheep or goat for 17 days prior to an embryo transfer procedure, wherein the continuous dose of progesterone is administered via a controlled internal drug release (CIDR) intravaginal device comprising 300 mg of progesterone;
administering a gonadotropin regimen to the sheep or goat 24 hours prior to the embryo transfer procedure, comprising a single dose of 200 IU of eCG and a single dose of 100 IU of hCG;
administering a single dose of prostaglandin to the sheep or goat 24 hours prior to the embryo transfer procedure, wherein the single dose of prostaglandin is selected from the group consisting of: 375 μg of Cloprostenol and 15 mg of Dinoprost; and
conducting the embryo transfer procedure, wherein a fertilized embryo is transferred into the sheep or goat to establish the pregnancy.

103. A method of establishing a pregnancy in a sheep or goat, the method comprising:
(i) stimulating follicular maturation in a donor sheep or goat by:
administering a continuous dose of progesterone to the donor sheep or goat for 17 days prior to harvesting an ovum from the donor sheep or goat, wherein the continuous dose of progesterone is administered via a controlled internal drug release (CIDR) intravaginal device comprising 300 mg of progesterone;
administering a releasing hormone regimen to the donor sheep or goat, comprising a single dose of 100 μg of gonadotropin releasing hormone (GnRH) administered 168 hours prior to harvesting the ovum from the donor sheep or goat;
administering a gonadotropin hormone regimen to the donor sheep or goat, comprising a continuous dose of 160 to 240 μg of follicle stimulating hormone (FSH) administered over a time period ranging from 120 to 24 hours prior to harvesting the ovum from the donor sheep or goat; and
administering a single dose of prostaglandin to the donor sheep or goat 5 days prior to harvesting the ovum from the donor sheep or goat, wherein the single dose of prostaglandin is selected from the group consisting of: 375 μg of Cloprostenol and 15 mg of Dinoprost;
(ii) synchronizing ovulation in the donor sheep or goat and a recipient sheep or goat by:
administering a continuous dose of progesterone to the recipient sheep or goat for 17 days prior to synchronization of ovulation, wherein the continuous dose of progesterone is administered via a controlled internal drug release (CIDR) intravaginal device comprising 300 mg of progesterone;
administering a gonadotropin regimen to the recipient sheep or goat 24 hours prior to synchronization of ovulation, comprising a single dose of 200 IU of eCG; and
administering a single dose of prostaglandin to the recipient sheep or goat 24 hours prior to synchronization of ovulation, wherein the single dose of prostaglandin is selected from the group consisting of: 375 μg of Cloprostenol and 15 mg of Dinoprost;
(iii) harvesting an ovum from the donor sheep or goat;
(iv) fertilizing the ovum to generate a fertilized embryo; and
(v) transferring the fertilized embryo into the recipient sheep or goat to establish a pregnancy.

104. A method of establishing a pregnancy in a sheep or goat, the method comprising:
(i) stimulating follicular maturation in a donor sheep or goat by:
administering a continuous dose of progesterone to the donor sheep or goat for 17 days prior to harvesting an ovum from the donor sheep or goat, wherein the continuous dose of progesterone is administered via a controlled internal drug release (CIDR) intravaginal device comprising 300 mg of progesterone;
administering a releasing hormone regimen to the donor sheep or goat, comprising a single dose of 100 μg of gonadotropin releasing hormone (GnRH) administered 168 hours prior to harvesting the ovum from the donor sheep or goat;
administering a gonadotropin hormone regimen to the donor sheep or goat, comprising a continuous dose of 160 to 240 μg of follicle stimulating hormone (FSH) administered over a time period ranging from 120 to 24 hours prior to harvesting the ovum from the donor sheep or goat; and
administering a single dose of prostaglandin to the donor sheep or goat 5 days prior to harvesting the ovum from the donor sheep or goat, wherein the single dose of prostaglandin is selected from the group consisting of: 375 μg of Cloprostenol and 15 mg of Dinoprost;
(ii) synchronizing ovulation in the donor sheep or goat and a recipient sheep or goat by:
administering a continuous dose of progesterone to the recipient sheep or goat for 17 days prior to synchronization of ovulation, wherein the continuous dose of progesterone is administered via a controlled internal drug release (CIDR) intravaginal device comprising 300 mg of progesterone;
administering a gonadotropin regimen to the recipient sheep or goat 24 hours prior to synchronization of ovulation, comprising a single dose of 100 IU of hCG; and
administering a single dose of prostaglandin to the recipient sheep or goat 24 hours prior to synchronization of ovulation, wherein the single dose of prostaglandin is selected from the group consisting of: 375 μg of Cloprostenol and 15 mg of Dinoprost;
(iii) harvesting an ovum from the donor sheep or goat;
(iv) fertilizing the ovum to generate a fertilized embryo; and
(v) transferring the fertilized embryo into the recipient sheep or goat to establish a pregnancy.

105. A method of establishing a pregnancy in a sheep or goat, the method comprising:
(i) stimulating follicular maturation in a donor sheep or goat by:
administering a continuous dose of progesterone to the donor sheep or goat for 17 days prior to harvesting an ovum from the donor sheep or goat, wherein the continuous dose of progesterone is administered via a controlled internal drug release (CIDR) intravaginal device comprising 300 mg of progesterone;
administering a releasing hormone regimen to the donor sheep or goat, comprising a single dose of 100 μg of gonadotropin releasing hormone (GnRH) administered 168 hours prior to harvesting the ovum from the donor sheep or goat;
administering a gonadotropin hormone regimen to the donor sheep or goat, comprising a continuous dose of 160 to 240 μg of follicle stimulating hormone (FSH) administered over a time period ranging from 120 to 24 hours prior to harvesting the ovum from the donor sheep or goat; and
administering a single dose of prostaglandin to the donor sheep or goat 5 days prior to harvesting the ovum from the donor sheep or goat, wherein the single dose of prostaglandin is selected from the group consisting of: 375 μg of Cloprostenol and 15 mg of Dinoprost;
(ii) synchronizing ovulation in the donor sheep or goat and a recipient sheep or goat by:
administering a continuous dose of progesterone to the recipient sheep or goat for 17 days prior to synchronization of ovulation, wherein the continuous dose of progesterone is administered via a controlled internal drug release (CIDR) intravaginal device comprising 300 mg of progesterone;
administering a gonadotropin regimen to the recipient sheep or goat 24 hours prior to synchronization of ovulation, comprising a single dose of 200 IU of eCG and a single dose of 100 IU or hCG; and
administering a single dose of prostaglandin to the recipient sheep or goat 24 hours prior to synchronization of ovulation, wherein the single dose of prostaglandin is selected from the group consisting of: 375 μg of Cloprostenol and 15 mg of Dinoprost;
(iii) harvesting an ovum from the donor sheep or goat;
(iv) fertilizing the ovum to generate a fertilized embryo; and
(v) transferring the fertilized embryo into the recipient sheep or goat to establish a pregnancy.

106. The method of any one of claims 101-105, wherein administering the gonadotropin hormone regimen to the donor sheep or goat comprises administering a continuous dose of 200 μg of FSH to the donor sheep or goat.

107. The method according to any one of claims 101-106, resulting in a pregnancy rate of 50% or greater in a plurality of recipient animals.

108. The method according to claim 107, resulting in a pregnancy rate in the plurality of recipient animals that ranges from 55% to 80%.

109. The method according to claim 108, resulting in a pregnancy rate in the plurality of recipient animals of 63.5%.
